(19) United States
(12) Patent Application Publication
(10) Pub. No.: US 2019/0151383 A1
(43) Pub. Date: May 23, 2019

(54) SIGNAL-SMART ONCOLYTIC VIRUSES IN TREATMENT OF HUMAN CANCERS

(71) Applicant: Faris Farassati, Overland Park, KS (US)

(72) Inventor: Faris Farassati, Overland Park, KS (US)

(21) Appl. No.: 16/198,578

(22) Filed: Nov. 21, 2018

Related U.S. Application Data
(60) Provisional application No. 62/589,325, filed on Nov. 21, 2017.

(51) Int. Cl. A61K 35/763; A61P 35/04; C12N 15/113; C12N 15/62; C12N 15/85; A61K 38/16; C07K 16/28; A61K 48/00

(52) U.S. Cl. CPC A61K 35/763; A61P 35/04; C12N 15/1135; A61K 48/0058; C12N 15/8509; A61K 38/162; C07K 16/28; C12N 15/62

(57) ABSTRACT

A recombinant lytic virus transcriptionally targeted against malignant cells. A promoter for a viral gene controlling replication is replaced with a promoter for a malignant factor such that the promoter for the malignant factor controls expression of the viral gene controlling replication. Accordingly, the recombinant lytic virus of the present invention only replicates within and kills cells expressing the malignant factor. In an embodiment, the recombinant lytic virus is a recombinant herpes simplex virus, and the viral gene controlling replication is a herpes alpha gene. In an embodiment, the recombinant lytic virus is transcriptionally targeted against cancer stem cells (CSCs). In an embodiment, the recombinant lytic virus is a recombinant herpes simplex virus-1 (HSV-1) with the promoter of CD133 controlling the expression of infected cell protein-4 (ICP4). In another embodiment, the recombinant lytic virus is a recombinant HSV-1 with the promoter of Ezh2 controlling the expression of ICP4.

### CROSS-REFERENCE TO RELATED APPLICATION

This application claims priority in U.S. Provisional Patent Application No. 62/589,325, filed Nov. 21, 2017, which is incorporated herein by reference.

### BACKGROUND OF THE INVENTION

### 1. Field of the Invention

The present invention relates generally to cancer treatments and, more specifically, to oncolytic viruses and methods of use thereof to target human cancer cells, including cancer stem cells and differentiated cancer cells, in the treatment of human malignancies.

### 2. Description of the Related Art

Cancer is one of the leading causes of death worldwide, with more than 1,700,000 new cancer cases and more than 600,000 cancer deaths projected to occur in the United States in 2018 alone. Treatments for many different types of cancers remain largely ineffective.

Cancer stem cells (CSCs), specifically, are of significant importance in cancer research due to their implications in understanding tumor biology as well as the development of novel cancer therapeutics. The theory of CSCs was first introduced in the 19th century. However, the possibilities for studying CSCs became available only in recent years.

The concept of CSCs has been portrayed by modeling tumors as being analogous to organs with a complicated and variant array of different cells that carry on with their specific assignments to maintain the structural and functional integrity of the tumor. Similar to the structure of normal tissues, according to this concept, tumors contain a subpopulation of cells with stem cell characteristics that are capable of self-renewal and differentiation. A large body of evidence now supports the validity of this theory. Due to their enhanced resistance to anti-cancer therapies, CSCs are considered to be responsible for the failure of many current cancer treatments, leading to the eventual growth of tumors after treatment. CSCs are also considered to be resistant to apoptosis, resulting in the eventual rise of tumors that are more unyielding to conventional chemotherapy.

Accordingly, treatments for effectively targeting and destroying CSCs appear to be extremely important areas of study for the development of successful human cancer treatments. A number of surface markers have previously been claimed to identify CSCs.

CD133, also known as prominin-1, is a glycoprotein which has been portrayed as one of the most important markers in CSCs involved in the biology of a number of human cancers, including liver, brain, colon, skin, and pancreas cancers. CD133 is expressed at very low levels in some normal human cells, however, CD13 expression is highly elevated in many tumors. For instance, elevated expression of CD133 has been identified in CSCs of medulloblastomas, glioblastomas, colorectal tumors, hepatocellular carcinomas, prostate cancers, melanomas, and malignant peripheral nerve sheath tumors. Additionally, circulating melanoma cells have been identified as CD133 expressing (CD133+) and indicative of a poor prognosis. Thus, CD133 appears to be an interesting target for anti-cancer treatment. However, heretofore, there have not been effective treatments to target and inhibit CD133 expressing (CD133+) cells as part of cancer treatment.

Furthermore, malignant primary brain and other central nervous system (CNS) tumors account for over 20,000 new cases of cancer in the United States every year, which amounted to 6.4 out of every 100,000 people from 2007 to 2011. Although this represents only a fraction of cancer incidences in the country, primary CNS tumors are very deadly. Over 14,000 people died of primary CNS tumors in 2014, which was more than 4 of every 100,000 people. About 40% of primary malignant CNS tumors are in a class of high grade glioma known as glioblastoma (GBM). Five year relative survival rates can be as low as 4% in certain age groups, with many living no more than 18 months after diagnosis. Since high grade gliomas are very difficult to treat, discovery of new methods of therapy is important to improve the outcomes of patients.

Polycomb group (PcG) proteins have been previously identified as repressors of HOX genes in fruit flies having mutant phenotypes involving posterior body segment development. Functionally, PcG proteins are classified according to their association with distinct multimeric complexes known as polycomb repressive complexes (PRC). PRCs play a major role in stem cell differentiation and transcriptional regulation during early embryonic development. A subgroup of PRC, named PRC2, includes Ezh2 (enhancer of zeste homologue 2), SUZ12 (suppressor of zeste 12), and EED (embryonic ectoderm development). These are involved in the initiation of epigenetic-mediated gene silencing. Ezh2 is the catalytically active component of PRC2 and involved in transcriptional repression of certain genes by trimethylation of lysine 27 and, to a lesser extent, lysine 9 of histone H3.

A range of human tumors have shown overexpression of Ezh2, and this overexpression has been associated with poor prognosis. PcG target genes are silenced in human tumors by DNA methylation of their promoter. The overall concept is that the PRC2 complex promotes tumorigenesis by inducing repression of tumor-suppressor genes, including the major tumor suppressor locus CDKN2A. Other studies suggest that an Ezh2-associated methyltransferase complex present in the cytoplasm may play a role in cellular adhesion and migration, thereby contributing to tumor dissemination as well.

For instance, studies have revealed that Ezh2 further plays a role in maintaining the undifferentiated characteristic nature of glioblastoma (GBM) stem cells (GSCs) by repressing the expression of basic metabolic panel (BMP) receptor 1B (BMPR1B). Moreover, the Oncomine database has revealed that four PcG genes (Ezh2, RBBP7, SUZ12, and YY1) are specifically expressed in brain tumors. Ezh2 expression has also been shown to increase along with increased tumor grade in both adult and pediatric brain tumors. Consistent with that finding, silencing Ezh2 has been shown to impair the self-renewal in vitro and the tumor-initiating capacity in vivo of GSCs. Treatment of GL261-GBM cells with EGF, βFGF, and serum withdrawal causes neurosphere formation and results in enhancement of Ezh2 expression. Comparisons have shown much higher expression of Ezh2 in GBM cells than in normal brain cells. Additionally, methylation of STAT3 has been shown to be a mechanism for involvement of Ezh2 in the biology of GSCs, resulting in promotion of tumorigenicity of GSCs. Using RNA interference, it was shown that down-regulation of Ezh2 expression in U87 human cells resulted in apoptosis and cell cycle arrest in G0/G1 phases, and similar data is observed for pediatric gliomas and medulloblastomas. Overall, data strongly suggest that Ezh2 is involved in important biological features of glioma.

Based on the foregoing, a strategy targeting Ezh2 seems to be a logical platform for anti-cancer agents. Heretofore, there have not been effective treatments to target and inhibit Ezh2 expressing (Ezh2+) cells.

Oncolytic viruses are a class of replication-competent viruses which replicate conditionally within cancer cells, leading to destruction or lysis of the cells. In many cases, oncolytic viruses have proven to be safe for use in humans in phase I clinical trials and in the case of Oncovex approved by the FDA for therapy of melanoma. However, cancer treatment utilizing oncolytic virus therapy is dependent on an ability of the virus to effectively infect and kill tumor cells while sparing normal cells. Heretofore, there have not been oncolytic viruses for targeting cancer cells, including targeting cancer stem cells, in cancer treatment with the advantages and features of the present invention.

### SUMMARY OF THE INVENTION

The present invention is based, inter alia, on a recombinant lytic virus transcriptionally targeted against malignant cells. The recombinant lytic virus is configured for replication and propagation of progeny of the lytic virus and for destruction of the targeted malignant cells. In constructing the virus of the present invention, the promoter for a controlling gene necessary for replication of the virus is replaced with the promoter for a known malignant factor. Thus, the recombinant virus of the present invention can only replicate in cells expressing the targeted malignant factor, or cancer marker. Replication of the lytic virus in the presence of the expressed malignant factor results in destruction of the malignant cell. In contrast, the recombinant virus of the present invention cannot replicate in cells that do not express the targeted malignant factor because the promoter necessary for replication is inactive without expression of the malignant factor.

In an aspect of the present invention, a novel, mutated version of herpes simplex virus-1 (HSV-1), referred to hereinafter as Signal-Smart 2 (SS2), is transcriptionally targeted against cells which express CD133 (CD133+). In an exemplary embodiment, a promoter for a herpes alpha gene, which is required for HSV-1 replication, is replaced with the promoter for CD133. In an exemplary embodiment, the replaced herpes alpha gene promoter is the promoter for infected cell protein-4 (ICP4), also known as the HSV-1 alpha-4 gene, which has a central role in gene expression of HSV-1 progeny.

In another aspect of the present invention, a novel, mutated version of HSV-1, referred to hereinafter as Signal-Smart 3 (SS3), is transcriptionally targeted against Ezh2 expressing (Ezh2+) cells. In an exemplary embodiment, a promoter for a herpes alpha gene, which is required for HSV-1 replication, is replaced with the promoter for Ezh2. In an exemplary embodiment, the replaced herpes alpha gene promoter is the promoter for infected cell protein-4 (ICP4), also known as the HSV-1 alpha-4 gene, which has a central role in gene expression of HSV-1 progeny.

Further aspects of the present invention include methods of treating cancer utilizing a recombinant oncolytic virus transcriptionally targeted against a pro-oncogenic promoter such as promoters of CSC markers, pro-oncogenic signaling pathways, transcription factors, and other malignancy promoting factors. Viruses embodying the present invention may be used as a preventative cancer treatment to prevent future tumor growth and/or as an inhibitive cancer treatment to inhibit growth of pre-established tumors. Furthermore, viruses of the present invention may be administered in a preventative manner to prevent the development of malignancies and/or prevent progression of malignancies. These viruses may be used to treat cancer(s) in humans and/or in other animals. Treatments utilizing viruses of the present invention may be introduced into a host via ingestion of pharmaceuticals, injection, infusion, instillation, inhalation, or any other method of medical introduction.

### BRIEF DESCRIPTION OF THE DRAWINGS

The drawings constitute a part of this specification and include exemplary embodiments of the present invention illustrating various objects and features thereof.

FIG. 1 is a tissue-level model of treating cancer stem cells (CSCs) with a virus embodying the present invention, resulting in destruction of CSCs and eventual tumor regression and elimination as the life cycles of differentiated cancer cells expire.

FIG. 2 is a cellular-level model illustrating the mechanism and structure of an embodiment of a recombinant oncolytic virus, Signal-Smart 2 (SS2), of the present invention with expression of ICP4 controlled by the promoter for CD133.

FIG. 3 is a cellular-level model illustrating the mechanism and structure of another embodiment of a recombinant oncolytic virus, Signal-Smart 3 (SS3), of the present invention with expression of ICP4 controlled by the promoter for Ezh2.

FIG. 4A is a schematic drawing of the structure of the SS2 virus.

FIG. 4B is a table showing confirmation of the structure of the SS2 virus by PCR. PCR reactions were used to confirm the structure of HSV-1 DNA polymerase in SS2 and HSV-1 infected Hep3B2 cells. The signal was negative for uninfected Hep3B2 cells. The backbone of the plasmid pTKCD133IDT was subjected to PCR in order to prove that SS2 preparation was free of contamination with this plasmid and validating other signals to originate from the viral genome and not the plasmid. The TK/CD133/ICP4 region was amplified by PCR and sequenced. The signal was positive for SS2 and plasmid but not for uninfected Hep3B2 cells. DsredExpress (DsRed), a part of pTKCD133IDT, was not found to be present in SS2. Due to deletion of DsredExpress, the fragment of ICP4/TK is smaller in SS2 compared to pTKCD133IDT.

FIG. 5 is an image showing the restriction digestion of genomic viral DNA by BamH1 obtained from analysis of SS2, d120, and HSV-1. The arrows refer to bands generated by digesting pTKCD133IDT using BamH1. No major difference in the viral genome of SS2 is observed as a result of genetic manipulation.

FIGS. 6A-6B shows graphs displaying the flow cytometry and percentages of CD133+ cells within cell lines of SK103, UACC257, human hepatocytes, Hep3B2, SW480, and Caco-2 cells and images illustrating the specificity of the SS2 virus for CD133+ cells compared to parental HSV-1.

FIG. 7 is imaging showing the production of viral proteins of cells infected with SS2 virus, evaluated by western blotting with a polyclonal antibody raised against mixed HSV-1 antigens. A significantly more comprehensive profile of viral proteins was observed in the case of cells with higher levels of CD133+ fraction (i.e., UACC257, Hep3B2, and Caco-2), as compared with cells with a lower level of CD133+ fraction (i.e., SW480 and SK103). Non-infected cells showed no significant bands.

FIG. 8 shows graphs and images illustrating loss of viability of CD133+ cells after infection with the SS2 virus compared with cells with low levels of CD133 expression.

FIGS. 9A-9C are graphs and images illustrating a loss of invasiveness of CD133+ cells after infection with the SS2 virus compared with cells with low levels of CD133 expression.

FIG. 10 shows graphs and imaging showing a loss of invasiveness for Caco-2 cells infected with the SS2 virus. FIG. 10 further shows no effect on invasiveness of SW480 cells having low levels of CD133 expression but significant decrease in invasiveness of SW480 cells having elevated CD133 expression when infected with the SS2 virus.

FIG. 11A shows a loss of viability in primary MP2 melanoma cells after exposure to the SS2 virus.

FIG. 11B is a graph showing increased apoptosis by cells infected with the SS2 virus based on the percentage of caspase activation.

FIG. 11C shows a graph and images illustrating a loss of invasiveness for primary MP2 melanoma cells after infection with the SS2 virus.

FIGS. 12A-12C are graphic representations of tumor sizes in vivo in male athymic nude mice of Hep3B2, Caco-2, and UACC257 cells exposed with the SS2 virus compared to a control.

FIGS. 13A-13C are graphic representations of melanoma tumor sizes in vivo in male athymic nude mice comparing tumors exposed to the SS2 virus with tumors not exposed to the SS2 virus, illustrating regression of pre-established melanoma tumors exposed to the SS2 virus.

FIG. 14A shows images showing CD133 expression after treatment with SS2 in melanoma tumor cells, illustrating replication of the SS2 virus. The arrowheads point to staining for CD133.

FIG. 14B shows images showing replication of the SS2 virus in pre-established melanoma tumors.

FIG. 15 shows western blot imaging illustrating a decrease in CD133 expression in both preventative and therapeutic mouse models after treatment with the SS2 virus. No changes in β-actin were observed.

FIG. 16 is a graph showing that viability of HepG2 cells is significantly reduced post-infection with the SS2 virus.

FIG. 17 is a graph showing that radiance (p/s/cm²/sr) of liver tumors over a 7-week treatment course is significantly higher for control-treated subjects as compared with SS2-treated subjects.

FIG. 18 shows a graph showing human alpha fetoprotein (AFP), an indicator of progression of the tumor, is significantly greater in the control group compared to the SS2-treated group.

FIG. 19 shows graphs illustrating the weight of male athymic nude mice over the course of treatment with the SS2 virus. Systemic injection of the SS2 virus does not appear to influence the growth of athymic nude mice.

FIG. 20 shows images showing no infection of vital organ cells with the SS2 virus as compared to CD133+ Hep3B2 cells.

FIG. 21A is a schematic drawing of the structure of the SS3 virus.

FIG. 21B is a table showing confirmation of the structure of the SS3 virus by PCR. PCR reactions were used to confirm the structure of HSV-1 DNA polymerase in SS3 and HSV-1 infected Vero cells. The signal was negative for uninfected Vero cells. The backbone of the plasmid pTEZH2IIDT was subjected to PCR in order to prove that SS3 preparation was free of contamination with this plasmid and validating other signals to originate from the viral genome and not the plasmid. The TK/Ezh2 region was amplified by PCR. The signal was positive for SS3 and plasmid but not for uninfected Vero cells.

FIG. 22 is an image showing the restriction digestion of genomic viral DNA by BamH1 obtained from analysis of SS3, d120, and HSV-1. No major difference in the viral genome of SS3 is observed as a result of genetic manipulation.

FIG. 23 shows western blotting imaging confirming high levels of Ezh2 expression in five well-studied glioma cell lines (Gli36, U87, U874, U118, and U251) but not in fetal human astrocytes (feHA).

FIG. 24 shows flow cytometry graphs illustrating high levels of Ezh2 expressing cells in glioma cell lines.

FIG. 25 is a graph showing that Ezh2 promoter was active in GBM cells, analyzed using a luciferase reporter assay, ranging from 2 to 5.8 folds as compared to the CMV promoter as control.

FIG. 26 is a graph showing that Ezh2 expression was found to be higher in side population (SP) glioma cells as compared to non-side population (non-SP) glioma cells.

FIG. 27 shows imaging showing that U87, U251, and U118 cells are permissive to the SS3 virus and to parental HSV-1.

FIG. 28 is western blot imaging showing the profile of SS3 viral proteins produced in GBM cell lines by using a polyclonal antibody raised against HSV-1 antigens. U87, U118, and U251 cell lines each produced SS3 proteins, while non-infected controls were void of viral proteins.

FIG. 29 shows proliferation assay graphs showing a significant reduction in GBM cell lines after infection with the SS3 virus.

FIG. 30 shows images illustrating signs of infection, such as rounding and clumping in Gli36 cells after infection with the SS3 virus. FIG. 30 further shows a graph illustrating significantly lower levels of radiance in U87Luc cells treated with the SS3 virus as compared with U87Luc cells not treated with the SS3 virus.

FIG. 31 shows graphs and images showing reduced invasiveness of U118 and U251 cells when infected with the SS3 virus.

FIG. 32 is a graph showing Ezh2 promoter activity in mouse glioma cells.

FIG. 33 shows flow cytometry graphs showing percentages of Ezh2 expressing cells in MC41-Cre, MC41-KRAS/Akt/Cre and MC41-EGFRvIII/Akt/Cre cell lines.

FIG. 34 shows graphs illustrating significant loss of viability in mouse glioma cells but not in background MC41-Cre cells because of lower promoter activity in those cells. FIG. 34 further shows morphology of MC41-Kras/Akt/Cre cells infected with the SS3 virus.

FIG. 35A shows images of subcutaneous tumor growth in vivo in male athymic nude mice and extracted tumors for control groups compared to SS3-treated groups. No tumor growth occurred in SS3-treated tests.

FIG. 35B shows the course of tumor growth in control-treated groups and the related volumes of the tumor.

FIG. 35C is a graph showing average tumor growth in control groups compared to SS3-treated groups (which showed no tumor growth).

FIG. 36 shows graphs illustrating regression of pre-established subcutaneous human tumors in athymic nude mice when treated with the SS3 virus.

FIG. 37 is a graph showing lower radiance levels for SS3-treated subjects compared to control-treated subjects in orthotopic intracranial tumors in athymic nude mice.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

Unless otherwise defined, all technical and scientific terms used herein have the same meaning as commonly understood by one of ordinary skill in the art of the invention. Methods and materials are described herein for use in the present invention, but other, suitable methods and materials known in the art can also be used. The materials, methods, and examples are illustrative only and not intended to be limiting. All publications, patent applications, patents, sequences, database entries, and other references mentioned herein are incorporated by reference in their entirety. However, in case of conflict, the present specification will control.

The present invention contemplates a novel, recombinant lytic virus configured for transcriptionally targeting and destroying cancer cells. Viruses which replicate via the lytic cycle cause destruction of an infected cell upon viral replication. Thus, a lytic virus can be very damaging to the health of a host organism, as the virus kills cells it infects and replicates within. However, if a lytic virus can be modified to only allow replication within, and thus destruction of, unwanted cells, such as malignant cells, such a modified lytic virus could be an effective mechanism for treatment to kill those unwanted cells. Accordingly, the present invention is a recombinant lytic virus which only permits replication in the presence of a known malignant factor promoter.

In order to selectively attack unwanted malignant cells, the recombinant virus of the present invention is transcriptionally targeted against cells having a known malignant factor or cancer stem cell marker, which is a biological substance, such as but not limited to a protein, known to be highly expressed within cancer cells. To accomplish this, the natural promoter for a gene which controls replication of the virus is replaced with a promoter for a cancer stem cell marker or other malignant factor highly expressed within malignant cells. With this arrangement, the new promoter for the controlling gene for replication of the virus requires expression of the targeted cancer stem cell marker or malignant factor to initiate transcription of the controlling gene. Accordingly, the virus can only replicate in cells expressing the cancer stem cell marker or malignant factor, and replication of the virus results in destruction of the cell within which it replicates.

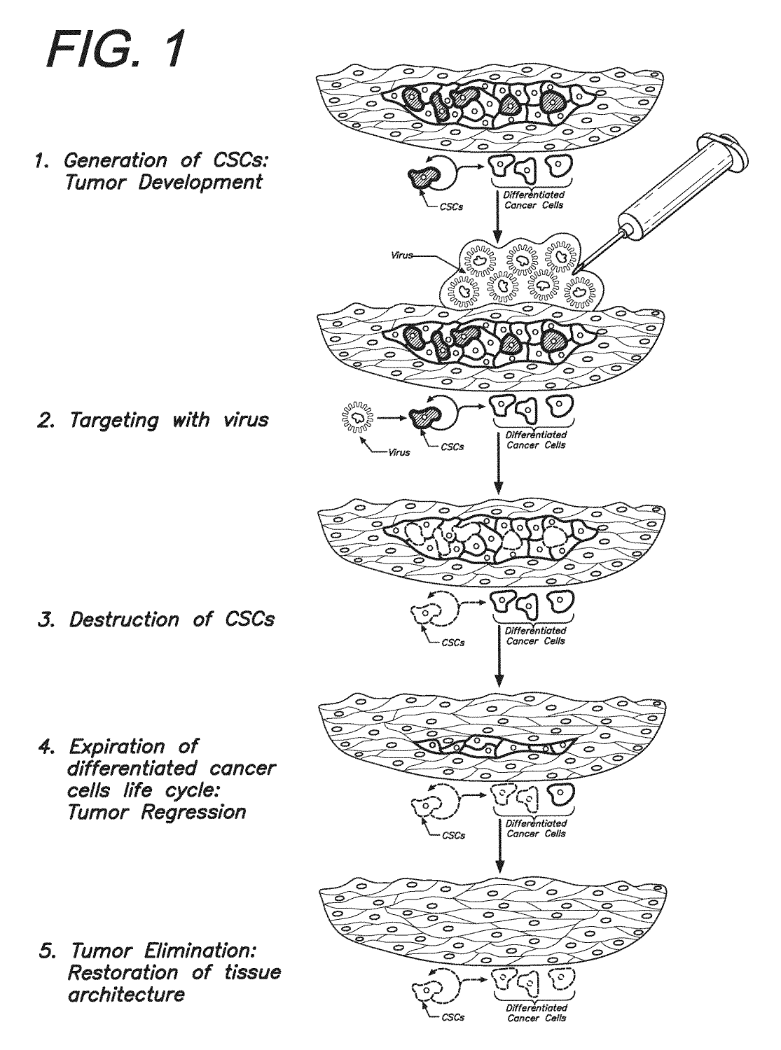

In an exemplary embodiment, a recombinant lytic virus of the present invention is used to transcriptionally target cells expressing a known cancer stem cell (CSC) marker. The concept of cancer stem cells, which is well-studied and well-supported by those skilled in the art, hinges on the theory that malignant tumors are structured similarly to organs, having an array of different cell types with specific assignments within the tumor. According to this theory, a subset of tumor cells, referred to as cancer stem cells (CSCs), show stem cell characteristics including being capable of self-renewal and differentiation. The motivation for targeting cancer stem cells is that if all cancer stem cells are killed, the tumor will no longer have cells which can self-renew and differentiate, and the tumor will regress as the life cycles of differentiated cancer cells expire. FIG. 1 illustrates the mechanism of targeting cancer stem cells with a virus embodying the present invention, resulting in tumor regression and eventually tumor elimination. The normal cell population can give rise to tumors due to the activation of CSCs by mechanisms that are not currently well understood, but may include pro-oncogenic activation and genomic instability. As CSCs replicate, they not only self-renew but they also give rise to differentiated cancer cells. Upon introduction of the virus of the present invention, the targeted CSCs become affected, resulting in their eventual elimination. Once the tumor's cell population is made up of only differentiated cancer cells because of the elimination of CSCs, the tumor cells will eventually complete their lifespan without the possibility of being renewed. This results in the elimination of tumor mass and the restoration of a normal tissue structure. While targeting cancer stem cells is one exemplary use of recombinant viruses of the present invention, it is not limiting. Other embodiments of the present invention may be configured to target differentiated cancer cells.

In embodiments, an oncolytic virus is used as the vehicle for composing a recombinant virus of the present invention. Any virus that can be transcriptionally regulated in terms of replication can be used as a vehicle for the present invention. Such embodiments may include, but are not limited to, members of the Human Herpes family, including but not limited to herpes simplex virus-1 (HSV-1) and herpes simplex virus-2 (HSV-2), adenoviruses, vaccinia virus, vesicular stomatitis virus, poliovirus, reovirus, senecavirus, RIGVIR, Semliki Forest virus, measles virus, Newcastle disease virus, coxsackie virus, Maraba virus, and retroviruses.

The virus is transcriptionally targeted against cancer cells by replacing the promoter for a gene which controls replication of the virus, such as immediate early genes, with the promoter for a malignant factor. In an exemplary embodiment, the oncolytic virus of the present invention is a recombinant HSV-1. In HSV-1, five proteins collectively referred to as herpes alpha genes, also known as class A genes, are immediate early genes most important in replication of HSV-1. These herpes alpha genes are controlled by promoters and are required for the herpes virus to replicate within an infected cell. Replacing the promoter for a herpes alpha gene with the promoter for a known cancer marker or malignant factor allows the mutated herpes virus to be transcriptionally targeted against cancer cells. In a preferred embodiment, the promoter for infected cell protein-4 (ICP4), also known as HSV-1 alpha-4 gene, is replaced with the promoter for a cancer marker. ICP4 controls of the expression of progeny of HSV-1 and is most important for HSV-1 to replicate. In alternative embodiments, promoters for herpes alpha genes other than ICP4, such as but not limited to infected cell protein-6 (ICP6), infected cell protein-3.54 (ICP34.5), and infected cell protein-27 (ICP27), may be replaced with the promoter for a cancer marker.

Researchers have identified many different cancer promoters, sometimes referred to as markers, malignant factors, or pro-oncogenic proteins, which are highly expressed within cancer cells. In the present invention, promoters of such cancer markers can be inserted within an oncolytic virus in place of the promoter for a viral gene controlling viral replication to transcriptionally target cancer cells with the oncolytic virus. Embodiments of recombinant viruses of the present invention can utilize any natural or synthetic sequences containing any elements of a cancer gene's promoters capable of driving the expression of a viral gene. Targets which have shown preferential expression in CSCs include surface markers such as CD24, CD34, CD38, CD117, CD44, CD90, CD133, CD271, ABCB5, and EpCAM, which have been shown to indicate a CSC subpopulation within a range of malignancies. Other target embodiments include pathways such as the JAK/STAT, Wnt/β-catenin, Hedgehog, Notch, TGF-β, Ral, and Ras pathways. Multidrug resistance pump ABC could be targeted or markers of microenvironment such as CXCL12/CXCR4 and VEGF/VEGFR. Further embodiments include promoters involved in expression of Cripto-1, Kpnb1, Enox-1, Enox-2, and β3 integrin and promoters containing Elk-1 binding factor. Additionally, transcription factors such as Oct4, sox2, nanog, Lin-29, KLf-4, c-myc and microRNAs specifically or preferentially expressed in CSCs could be transcriptionally targeted with a virus of the present invention.

The present invention further covers recombinant lytic viruses as described above further coated with polymers, nanomers, or any combination thereof to improve the efficiency of targeting cells expressing CSC stem cells or other malignant factors. The present invention also covers recombinant lytic viruses as described above further equipped with antibodies, aptamers, receptors, homing devices, or any combinations thereof to improve the efficiency of targeting cells expressing CSC stem cells or other malignant factors.

### II. Signal-Smart 2 (SS2) Virus

In an exemplary embodiment of the present invention, a recombinant HSV-1, referred to herein as Signa-Smart 2 (SS2) virus, is used to target cells expressing CD133. CD133, also referred to as prominin-1, is a surface glycoprotein which has been identified as an important marker of CSCs in many different types of malignancies. CD133 has been shown to be highly expressed in CSCs in medulloblastomas, glioblastomas, colorectal tumors, hepatocellular carcinomas, prostate cancers, melanomas, and malignant peripheral nerve sheath tumors. Circulating melanoma cells have also been identified as CD133 expressing and indicative of poor prognosis. While CD133 is expressed in low levels in differentiated cancer cells and in some other human cells, its highest expression has been shown to be in CSCs, making CD133 expressing cells an effective target for the present invention.

In this embodiment, the HSV-1 virus is engineered to insert the promoter for CD133 within the virus in place of the promoter for infected cell protein-4 (ICP4) such that the expression of ICP4 is controlled by the promoter of CD133. The CD133 promoter is activated in the presence of expressed CD133. With such an arrangement, ICP4, which controls the expression of HSV-1 progeny and is required for replication of HSV-1, is only expressed in cells which express CD133. Accordingly, this embodiment of a recombinant HSV-1 virus is configured for selectively replicating within and killing cells which express CD133.

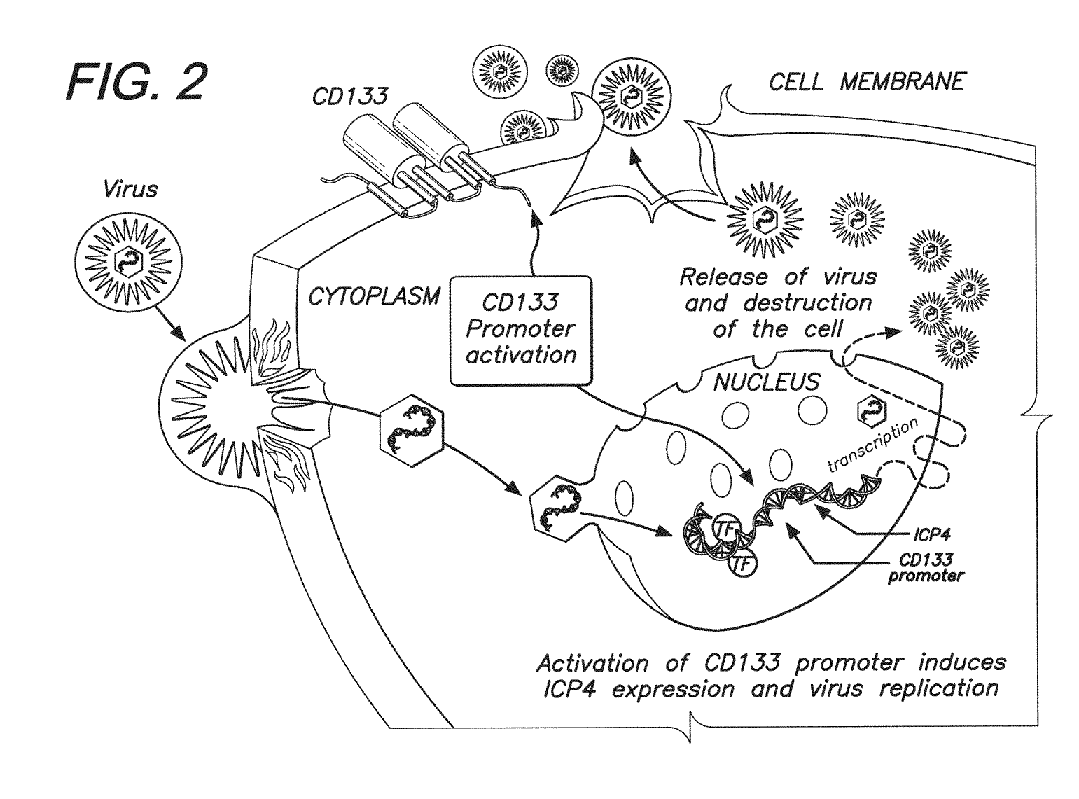

FIG. 2 is a cellular level diagram showing the mechanism of transcriptionally targeting and killing CD133 expressing cells with the SS2 virus. The virus enters the cell, and the viral DNA enters the nucleus. Expression of CD133 within the cell activates the CD133 promoter, which then expresses the ICP4 gene, allowing replication of the virus. Progeny of the virus are produced and released, resulting in destruction of the cell and membrane.

To construct a plasmid containing the ICP4 gene controlled by the CD133 promoter, the CD133 promoter was amplified using polymerase chain reaction (PCR) from human genomic DNA, isolated from a cultured Hep3B2 cell. Hep3B2 cells are hepatocellular carcinoma cells which have shown to express high levels of CD133. The CD133 promoter was isolated from utilizing sense and antisense primers having added AgeI and SpeI sites, respectively.

The pTIIDT plasmid was constructed as described by Pan et al. in Utilizing ras signaling pathway to direct selective replication of herpes simplex virus-1, PLoS One, 2009; 4:e6514, which is incorporated by reference herein in its entirety. The ICP4 gene was amplified from DNA template pGH108 using PCR. The PCR product was inserted into the vector pCR-Blunt-TOPO (Invitrogen, catalog no: K2800-20), and an EcoRI-ICP4-EcoRI fragment was isolated and subcloned into the vector pIRES/DsRed express (Clontech, catalog no: 632463) between CMV and IRES/DsRed genes at the EcoRI single site. This construct is referred to as pIID. Part of the human thymidine kinase (TK) gene was amplified from DNA template pHSV106 using PCR, and the PCR product was then cloned into the vector pCRII-TOPO (Invitrogen, catalog no: K4600-01). A NheI-TK-NheI fragment, TK5′, from the TOPO-clone was subcloned into the plasmid pIID, upstream of the ICP4 gene. This construct was designated as pTIID. The 3′ segment of the viral TK gene was also amplified from pHSV106 using PCR and subcloned into pCRII-TOPO. A HpaI-TK-HpaI fragment, TK3′, was cloned into the plasmid, pTIID, on the 3′ of the IRES/DsRed genes at its HpaI single site. This construct was designated as pTIIDT.

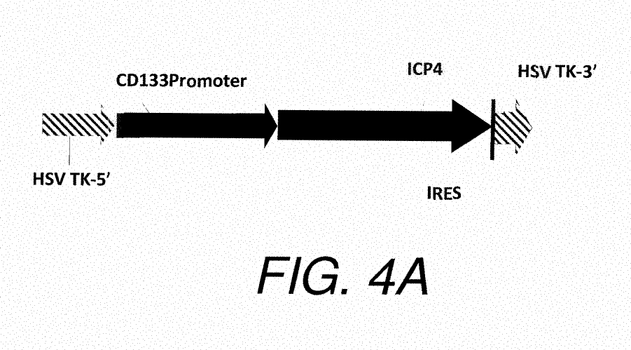

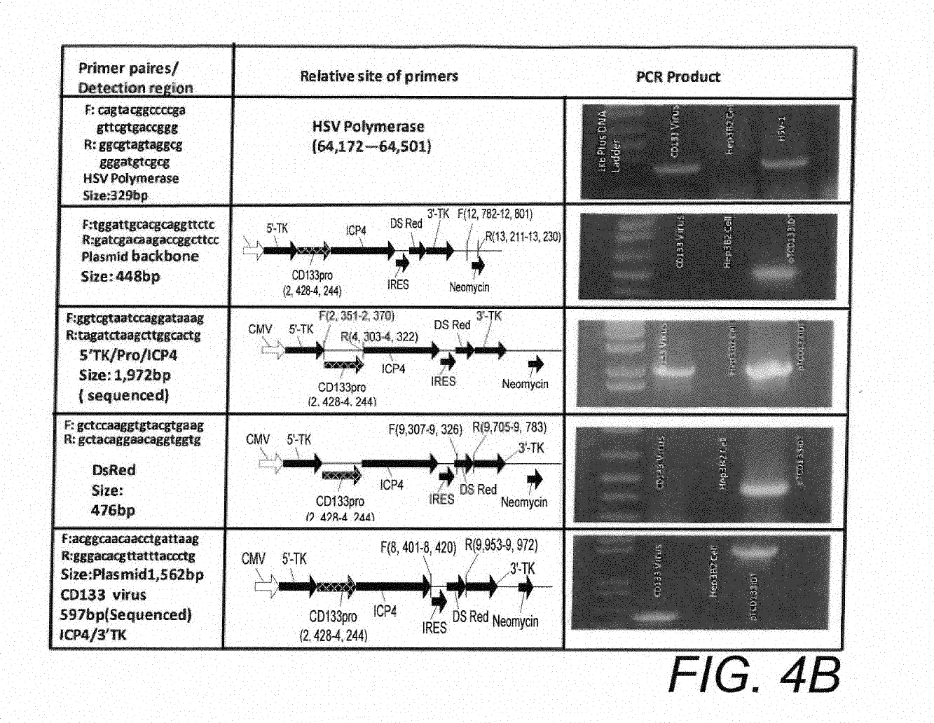

The CD133 PCR fragment was inserted into the pTIIDT plasmid by homologous recombination via AgeI and SpeI restriction sites. This resulting plasmid is referred to as pTKCD133IDT, the structure of which is shown in FIG. 4A.

E5 cells were grown in vitro and transfected with pTKCD133IDT using Liprofectamine 2000 (Invitrogen, CA). The cells were infected with the d120 virus 72 hours later. The d120 virus is a mutant HSV-1 void of ICP4-genes, which can produce a recombinant virus once the ICP4 gene is provided in trans by E5 cells. Three days after pTKCD133IDT infected with d120 virus, the virus was harvested. CD133-rich Hep3B2 cells were used for recombinant virus purification to eliminate ICP4 negative virus. The virus was then serially diluted, and a sample from each dilution was tested with Hep3B2 cells. The well with the highest dilution and positive virus signal was determined to contain the isolated virus, named Signal-Smart 2 (SS2) virus. The genomic composition of the recombinant virus was identified and confirmed by different PCR primer pairs (see FIG. 4B).

Experiments have shown the SS2 oncolytic virus to preferentially target CD133 expressing cells, particularly when compared to infection by parental HSV-1. Furthermore, the SS2 virus has shown a loss of viability for cells it infects. Reduction in tumor cell population by SS2 virus appears more prominent at later time points, such as five days or more post-infection. This is in contrast to parental HSV-1, which typically kills cells within the first 24 hours post-infection. Further experimentation has shown that infection with SS2 virus has decreased invasiveness of cells cancer cells expressing CD133 and induced apoptosis. The SS2 virus has been shown to inhibit tumor growth in mice, cause tumor regression in mice, and cause tumor regression in an orthotopic mouse hepatocellular carcinoma (HCC) model. Moreover, the SS2 virus did not appear to infect vital, non-malignant organs in mice.

Tests have shown the SS2 virus to be effective in treatments against hepatocellular carcinoma (liver cancer) colorectal cancers, and melanoma (skin cancer), but SS2 appears likely to be effective for treating any cancer cells which highly express CD133. Treatment with SS2 virus could be conducted via a pharmaceutical composition containing the SS2 virus, injection, infusion, instillation, inhalation or any other form of medical administration. SS2 virus may be used to preferentially target CSCs and/or differentiated cancer cells expressing CD133 in humans or in other mammals.

### III. Signal-Smart 3 (SS3) Virus

In another exemplary embodiment of the present invention, a recombinant HSV-1, referred to herein as Signal-Smart 3 (SS3) virus, is used to target cells expressing Ezh2. A range of malignancies have shown overexpression of Exh2. For instance, Ezh2 is overexpressed in differentiated glioblastoma (GBM) cells and is also highly involved in the biology of GBM stem cells (GSCs). The expression of Ezh2 in a number of well-studied glioma cell lines is shown to be elevated in terms of the amount of protein expressed and the subpopulation of cells positive for Ezh2. GSCs, as studied by sorting for SP cells, were found to be high in Ezh2 expression as compared with non-SP cells. GSCs are proposed to be responsible for not only replenishing the tumor population but also for resistance to chemotherapy, so the ability to target this subpopulation with the SS3 virus is promising. Promoter activity is also universally high in the cell lines that were tested, showing that Ezh2 is readily permissive to the SS3 virus.

In this embodiment, the HSV-1 virus is engineered to insert the promoter for Ezh2 within the virus in place of the promoter for infected cell protein-4 (ICP4) such that the expression of ICP4 is controlled by the promoter of Ezh2. The Ezh2 promoter is activated in the presence of expressed Ezh2. With such an arrangement, ICP4, which controls the expression of HSV-1 progeny and is required for replication of HSV-1, is only expressed in cells which express Ezh2. Accordingly, this embodiment of a recombinant HSV-1 virus is configured for selectively replicating within and killing cells which express Ezh2.

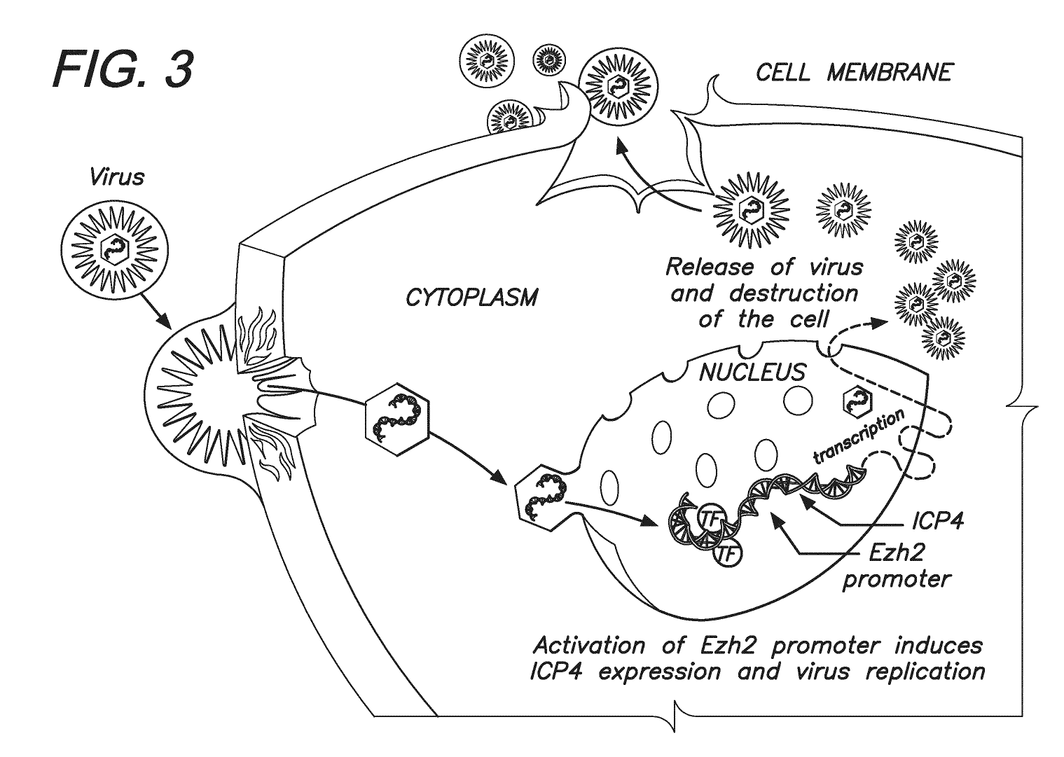

FIG. 3 is a cellular level diagram showing the mechanism of transcriptionally targeting and killing Ezh2 expressing cells with the SS3 virus. The virus enters the cell, and the viral DNA enters the nucleus. Expression of Ezh2 within the cell activates the Ezh2 promoter, which then expresses the ICP4 gene, allowing replication of the virus. Progeny of the virus are produced and released, resulting in destruction of the cell and membrane.

To construct a plasmid containing the ICP4 gene controlled by the Ezh2 promoter, the Ezh2 promoter was amplified using polymerase chain reaction (PCR) from human genomic DNA, purified from a cultured NCCIT cell. NCCIT cells are embryonic carcinoma cells which express high levels of Ezh2. The Ezh2 promoter was isolated from utilizing sense and antisense primers having added AgeI and SpeI sites, respectively.

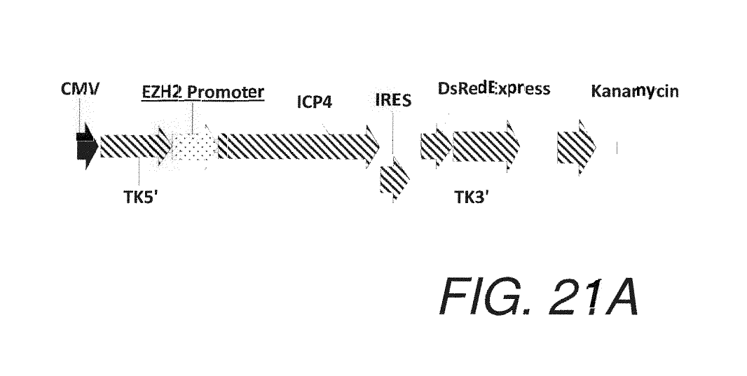

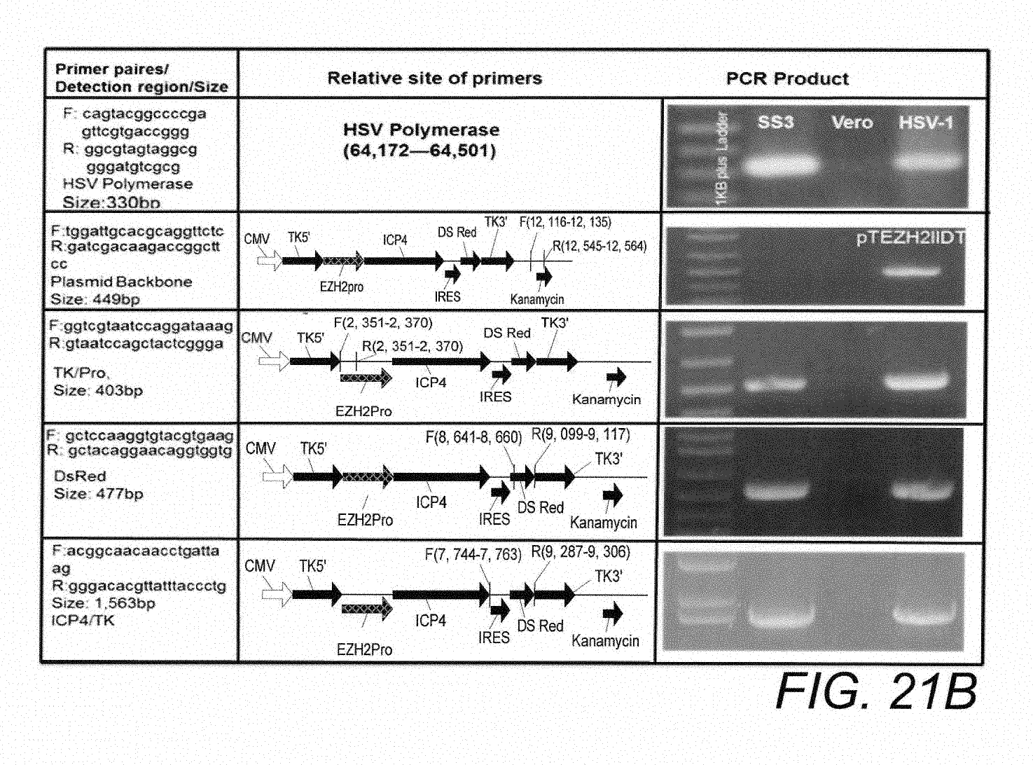

The Ezh2 PCR fragment was inserted into the pTIIDT plasmid, as described above, by homologous recombination via AgeI and SpeI restriction sites. This resulting plasmid is referred to as pTEZH2IIDT, the structure of which is shown in FIG. 21A.

E5 cells were grown in vitro and transfected with pTEZH2IIDT using Liprofectamine 2000 (Invitrogen, CA). The cells were infected with the d120 virus, as described above, 72 hours later. Three days after pTEZH2IIDT infected with d120 virus, the virus was harvested. Vero cells were used for recombinant virus purification to eliminate ICP4 negative virus. The virus was then serially diluted, and a sample from each dilution was tested with Vero cells. The well with the highest dilution and positive virus signal was determined to contain the isolated virus, named Signal-Smart 3 (SS3) virus. The genomic composition of the recombinant virus was identified and confirmed by different PCR primer pairs (see FIG. 21B).

Experiments have shown the SS3 oncolytic virus to preferentially target Ezh2 expressing cells and reduce their proliferation and invasiveness. The SS3 virus has further been shown to inhibit tumor growth in mice, cause tumor regression in mice, and cause tumor regression in an orthotopic mouse brain tumor model.

Tests have shown the SS3 virus to be effective in treatments against glioma cells expressing Ezh2, but SS3 appears likely to be effective for treating any cancer cells which highly express Ezh2. SS3 virus may be used to preferentially target glioblastoma stem cells (GSCs) and/or differentiated cancer cells expressing Ezh2 in humans or in other mammals.

There are a number of possibilities for delivery of the SS3 virus to the site of brain tumors, including methods with and without disruption of the blood-brain barrier. In animal models, an osmotic pump to continuously deliver the virus may be a possibility for convective delivery. Subcutaneous reservoirs with catheters to the tumor bed are possible modes of reintroducing the virus over the postoperative course. Placement of the virus following a laser interstitial thermal therapy (LITT) treatment may also hold promise with a compromise in blood brain barrier. Additionally, novel polymers that hold the virus against the tumor bed wall and dissolve over time hold promise for placing the virus in closest proximity to neoplastic cells. Such advancements in pharmacokinetics of oncolytic viruses enhance their potentials to meet current clinical needs. Effective and specific versions of viruses, such as SS3, which are designed on the basis of cell signaling features of cancer cells can serve as attractive tools for therapy of brain tumors.

Additional embodiments of the present invention include any chemical modification of the viruses, such as coating the virus with polymers or nanomers to improve the efficiency, specificity, or biodistribution of the virus. Moreover, any chemical modification of these viruses to include antibodies, monoclonal antibodies, or natural or synthetic receptors or aptamers further comprise alternative embodiments of the present invention.

It is to be understood that while certain aspects, embodiments, and examples of the invention are described and shown herein, the invention is not limited thereto and can assume a wide range of other, alternative aspects, examples, and embodiments.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art of the invention may develop equivalent means or reactants without the exercise of inventive capacity and without departing from the scope of the invention.

All values within these examples are expressed as mean±standard deviation (SD) and were analyzed using the two-tailed Student's t-test. p<0.05 was considered significant. The α-value was set at 0.05.

### Example 1

### Construction of Plasmid Containing ICP4 Gene Controlled by CD133 Promoter

The plasmid pTIIDT, previously described by Pan et al. in Utilizing ras signaling pathway to direct selective replication of herpes simplex virus-1, PLoS One, 2009; 4:e6514, which is incorporated by reference herein in its entirety, was constructed as follows. Using a sense primer, 5′-ctcacagctagcttgggtaacgccagggttttc-3′ and an antisense primer, 5′-actgtatagatctcgcccctcgaataaacaacgc-3′, the ICP4 gene was amplified by PCR from DNA template, pGH108. The PCR product (4.2 KB) was then inserted into the vector, pCR-Blunt-TOPO (Invitrogen, catalog no: K2800-20). An EcoRI-ICP4-EcoRI fragment from the TOPO clone was then isolated and subcloned into the vector, pIRES/DsRed express (Clontech, catalog no: 632463), between CMV and IRES/DsRED genes at EcoRI single site. The construct was designated as pIID. Next, part of human thymidine kinase (TK) gene was amplified from DNA template, pHSV106, by using primers, 5′-atcgctagctccaagactgacacatt-3′ and 5′-atgctagcactagtaccggtagtactgctgaggtgggctttggacgtctt-3′. The PCR product was then cloned into the vector, pCRII-TOPO (Invitrogen, catalog no: K4600-01). A NheI-TK-NheI fragment, TK5′, from the TOPO-clone was then subcloned into the plasmid, pIID, in front of the ICP4 gene. The construct was designated as pTIID. The 3′ segment of the viral TK gene was also amplified from pHSV106 by primers 5′-gggttaacatttaaatcaggtcgccgttgggggcca-3′ and 5′-gggttaacaaatgagtcttcggacctc-3′ and subcloned into the pCRII-TOPO as mentioned above. The HpaI-TK-HpaI fragment, TK3′, was cloned into the plasmid, pTIID, on the 3′ of the IRES/DsRed genes at its HpaI single site. This construct was designated as pTIIDT.

A 1.8 kb fragment containing the CD133 promoter was amplified by nested PCR from human genomic DNA isolated from cultured Hep3B2 cells. The first round of PCR was performed using forward primer 5′-aaactgtctttcctggcttc-3′ and reverse 5′-ttccttaaacatactcaccg-3′. The nested PCR was performed using forward primers 5′-tgaaccggtcctgcaagcggcacatcagag-3′ (AgeI site, underlined, was added to this primer) and reverse primer 5′-tgaactagtgcgttagcatcgctttaattcag-3′ (SpeI site, underlined, was added to this primer). The PCR fragment containing the CD133 promoter was inserted into the plasmid pTIIDT (Pan et al.), via AgeI and SpeI restriction sites and flanked by ˜1 kb sequence of HSV-1 thymidine kinase (TK) DNA. The resulting plasmid was named pTKCD133IDT.

### Example 2

### Generation of the SS2 Virus, an Oncolytic HSV-1 Targeting CD133+ Cells

E5 cells grown on a 150 mm dish to −80% confluency were transfected with pTKCD133IDT using Lipofectamine 2000 (Invitrogen, CA, USA) according to the manufacturer's instructions. Transfected cells were infected with d120 virus 72 h later. The d120 virus is a mutant HSV-1 void of ICP4-genes, which can produce a recombinant virus once the ICP4 gene is provided in trans by E5 cells, described by Su et al. in Evidence that the immediate-early gene product ICP4 is necessary for the genome of herpes simplex virus type 1 ICP4 deletion mutant strain d120 to circularize in infected cells, J Virol, 2006; 80:11589-11597; by DeLuca et al. in Isolation and characterization of deletion mutants of herpes simplex virus type 1 in the gene encoding immediate-early regulatory protein ICP4, J Virol, 1985; 56:558-570; and by Wu et al. in Prolonged gene expression and cell survival after infection by a herpes simplex virus mutant defective in the immediate-early genes encoding ICP4, ICP27, and ICP22, J Virol, 1996; 70:6358-6369, each of which is incorporated by reference in its entirety. In this stage, recombination of the plasmid with genomic viral DNA (HSV-1 TK with insertion of CD133-promoter-ICP4 gene) results in insertion of recombinant ICP4 gene in the HSV-1 TK gene of the d120 virus and production of the recombinant virus expressing ICP4 under control of the CD133 promoter. The supernatant containing the virus was harvested 3 days later.

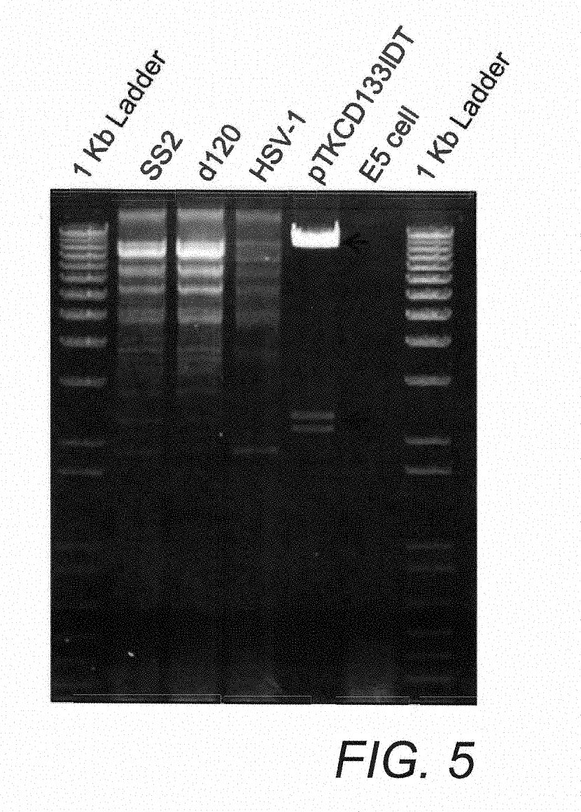

Next, Hep3B2 (hepatocellular carcinoma expressing high levels of CD133) was used for further amplification and purification of the recombinant virus. Briefly, the virus was passed in Hep3B2 cells for three rounds to eliminate any ICP4-negative variants. The last batch of virus was then serially diluted (10⁻¹ to 10⁻⁹), and a volume of 500 μl from each dilution was added to the duplicate wells of a 24-well plate containing Hep3B2 cells at ˜80% confluency. The culture was harvested 4 days later. The well with the highest dilution and positive virus signal, determined with PCR for HSV-1 polymerase, was considered to contain the isolated recombinant virus. Using PCR by different primer pairs (FIG. 4B), the genomic composition of this recombinant virus was identified and confirmed, and the titer was determined to be ˜10⁷ plaque-forming units. This resulting virus was named the Signal-Smart 2 (SS2) virus. The restriction map of viral genomic DNA extracted from SS2-infected Hep3B2 cells was compared with viral DNA from d120 infected E5 cells, as well as Vero cells infected with the parental HSV-1 (FIG. 5). The backbone plasmid pTKCD133IDT was also subjected to digestion by BamH1. After digestion, a similar pattern was observed for SS2, d120, and HSV-1, thus indicating that insertion of the recombinant ICP4 gene did not result in unwanted alteration in the structure of the viral genome. The arrows in FIG. 5 show the fractions that resulted from digestion of the plasmid pTKCD133IDT.

The oncolytic SS2 virus was constructed on the basis of the d120 system as described above. The virus was purified, characterized, and concentrated; and stocks were stored in a −80° C. freezer in 3% autoclaved non-fat milk. Viral titration was assessed by immunofluorescence microscopy as well as polymerase chain reaction (PCR) and was determined to be ˜10⁷ plaque forming unit (pfu)/ml.

### Example 3

### SS2 Oncolytic Virus Preferentially Targets CD133+ Cells

In order to study the specificity of the SS2 virus against CD133+ cancer stem cells, three different models of cancer cells that contained a CD133+ subpopulation were tested. Cell lines UACC257 (melanoma), Caco-2 (colorectal cancer), and Hep3B2 (hepatocellular carcinoma) were used as cells with high levels of CD133 for this study. UACC257 contained ˜6% CD133+ cells, while Hep3B2 and Caco-2 contained ˜80% CD133+ cells. Cells with lower percentages of CD133 expression (<1-3%) included melanoma SK103, SW480 colorectal cancer, and human hepatocytes. Each of these cells were infected with the SS2 virus and multiplicity of infection pictures (MOI˜3) were taken at 48 h post-infection.

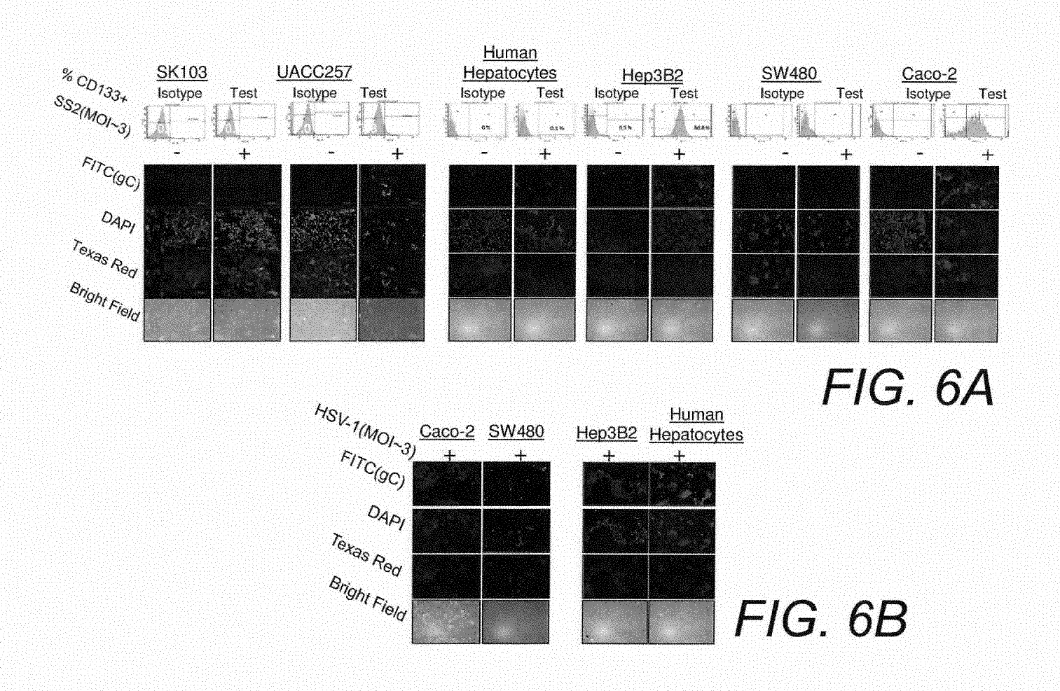

The cells were also subjected to immunofluorescence studies using a monoclonal antibody against glycoprotein-C (gC), a global standard for detection of virus infection and replication. For immunofluorescence analysis, different cells were grown in 8-well slide chambers (Falcon, Calif., USA) and infected with SS2 or HSV-1 (strain F), or mock-infected using 3% autoclaved milk. At different times post-infection, cells were fixed in 100% acetone for 10-15 minutes and left at room temperature to dry before incubation with a fluorescin-labeled mouse monoclonal antibody against HSV-1 gC antigen (Labvision, MI, USA) for 30 min at 37° C. The anti-gC antibody preparation contained Texas Red stain as well. The slides were then washed with distilled water, dried, mounted using a media containing DAPI (4′, 6-diamidino-2-phenylindole), viewed with an inverted Olympus DP71 and an attached computer, and processed with appropriate software. Cells were also photographed for their morphology using light microscopy. FIG. 6A displays the results of exposure of different cells to SS2, showing the virus' preferential capability to cause infection in UACC257 cells but not in SK103 cells. The upper row of FIG. 6A portrays the results of flow cytometry and the percentage of CD133+ cells for each cell line at the time of infection. For flow cytometry analysis, cancer cells were collected with non-enzymatic cell dissociation solution (Sigma, MO, USA) from culture dishes and washed with phosphate buffered saline (PBS). Washed cells were incubated with anti-CD133-APC antibody and FcR blocking reagent (Miltenyi Biotec, MA, USA) for 10 min on ice. After washing and staining cells with PBS twice, CD133+ cells were analyzed with a LSR II instrument (BD Biosciences). Staining with mouse IgG2b-APC was used as an isotype control. Staining for DAPI (nuclear DNA), Texas Red (cytoplasm), and bright field microscopy (cell morphology) are also shown. A similar phenomenon was observed once Hep3B2 and Caco-2 were exposed to the SS2 (FIG. 6A). In both cases, cells with a higher fraction of CD133+ cells (Hep3B2 and Caco-2) were significantly more permissive to the SS2 virus when compared to cells with a lower percentage of CD133+ subpopulation, i.e., human hepatocytes and SW480 cells. The parental HSV-1, however, lacked the capability to differentiate between high and low CD133+ cells and caused comparable infection in these cells (FIG. 6B).

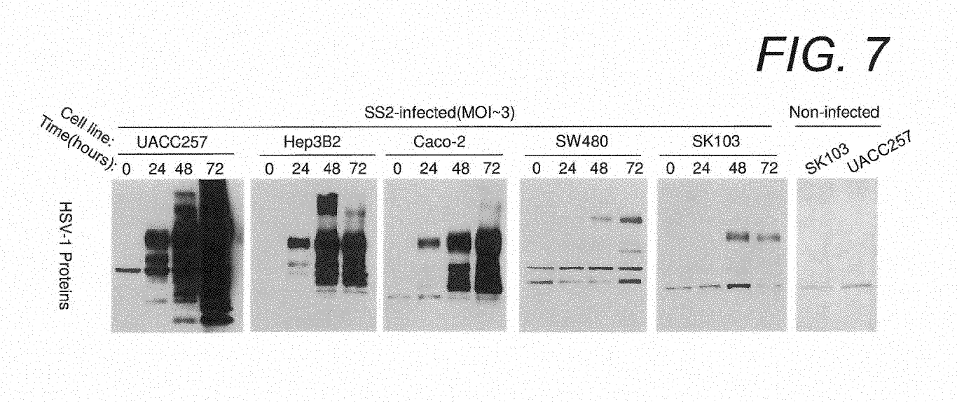

Another important measure of permissiveness to SS2, herpetic protein synthesis, was evaluated by western blotting (FIG. 7). In this experiment, the production of SS2 viral proteins was evaluated using an antibody raised against a mixture of HSV-1 antigens. An increased rate of viral protein production was observed at 48 and 72 h post-infection for UACC257, Hep3B2, and Caco-2 cells as compared to SW480 or SK103 cells. Non-infected SK103 and UACC257 cells were mainly void of bands, thus indicating the antibody's specificity for herpes proteins. In the same manner, immediate lysis of cells after the addition of the virus, represented by time point zero, did not generate a substantial profile for viral proteins due to lack of viral replication.

### Example 4

### Infection with SS2 Oncolytic Virus Results in Loss of Viability

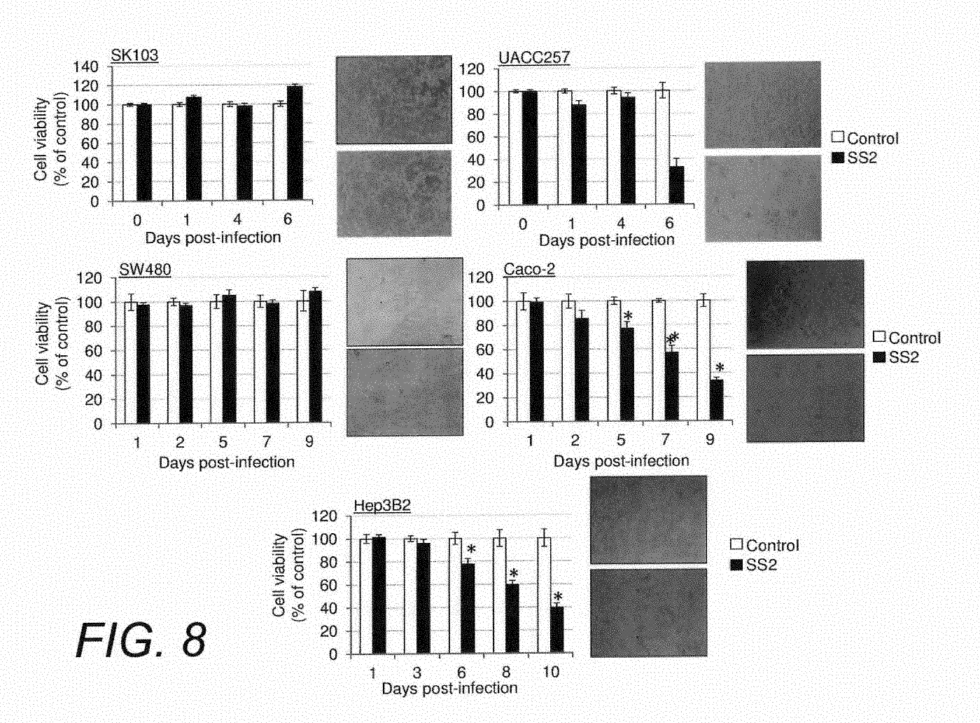

In the next step, we evaluated the proliferation of cancer cells following exposure to the SS2 virus (MOI˜2). Cells were plated in 96-well plates and cultured in growth medium overnight. On the following day, the cells were infected with viral particles or 3% autoclaved non-fat milk as a control. At the indicated time points, the proliferation assay was performed using the WST-1 assay (Millipore, MA, USA). As can be seen in FIG. 8, the virus produced a significant decrease in the proliferation rate of UACC257 at long time points (e.g., day 6), while SK103 cells were not affected. The morphology panels in FIG. 8 show dissemination of UACC257 monolayer, but not SK103, at day 6 post-infection due to exposure to SS2. A similar pattern was seen for colorectal cancer cells. The rate of Caco-2 proliferation subsided at day 5 post-infection, while no major change was observed for SW480 cells. Finally, Hep3B2 cells behaved similarly in terms of loss of proliferative capabilities starting at day 6 post-infection.

### Example 5

### Infection with SS2 Oncolytic Virus Results in Loss of Invasiveness

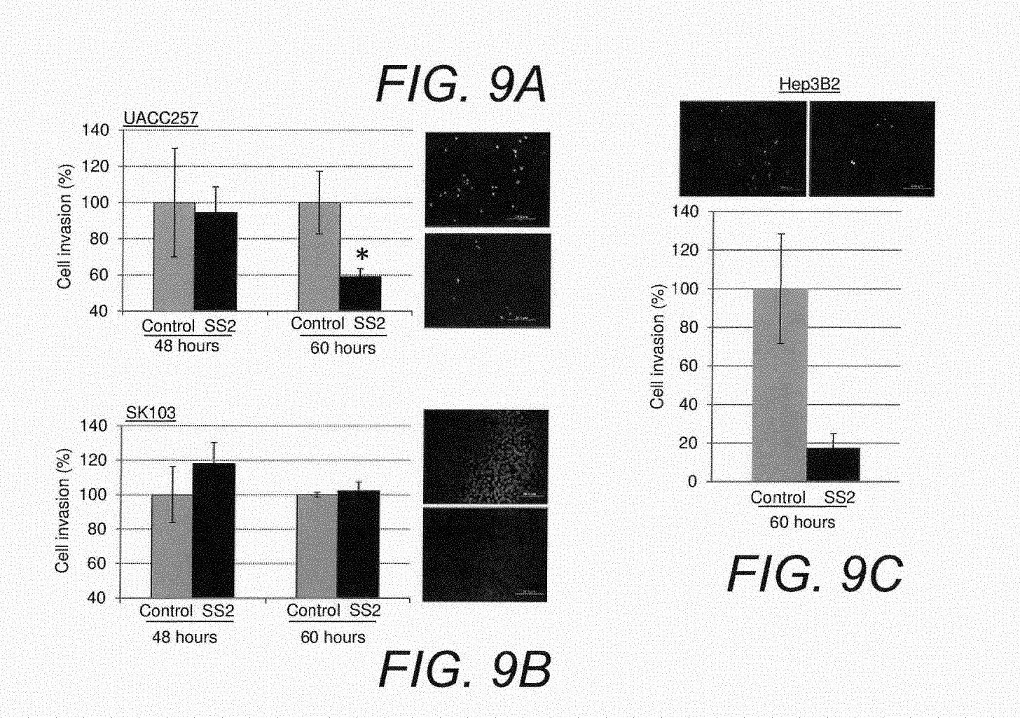

In the next step, the in vitro invasiveness of infected cells was evaluated using a modified Boyden chamber assay. The cell invasion assay was performed using a BioCoat Matrigel Invasion Chamber (BD Biosciences, MD, USA). Cancer cells were seeded into Matrigel-coated inserts fitting 24-well plates. After 8 h, cells were infected with SS2 at MOI˜2-3. As the cells infiltrated through the layer of Matrigel in the next 24 h, the fraction of invaded cells attached to the bottom disks were detected by fixing them with 100% methanol and placing them on a slide glass with mounting medium containing 4′,6-diamidino-2-phenylindole (DAPI). Quantification was performed by counting the DAPI-stained nuclei on this disk under a fluorescence microscope. Once UACC257 and SK103 cells were infected with SS2 (MOI˜3), a significant reduction in invasiveness of UACC257 cells was seen at 60 h post-infection (FIG. 9A), while the invasiveness of SK103 cells was not markedly altered (FIG. 9B). Hep3B2 cells behaved similarly after exposure to the SS2 virus with an 80% decrease in invasiveness of the infected cells (FIG. 9C). FIGS. 9A, 9B and 9C show DAPI-stained nuclei of the invaded cells.

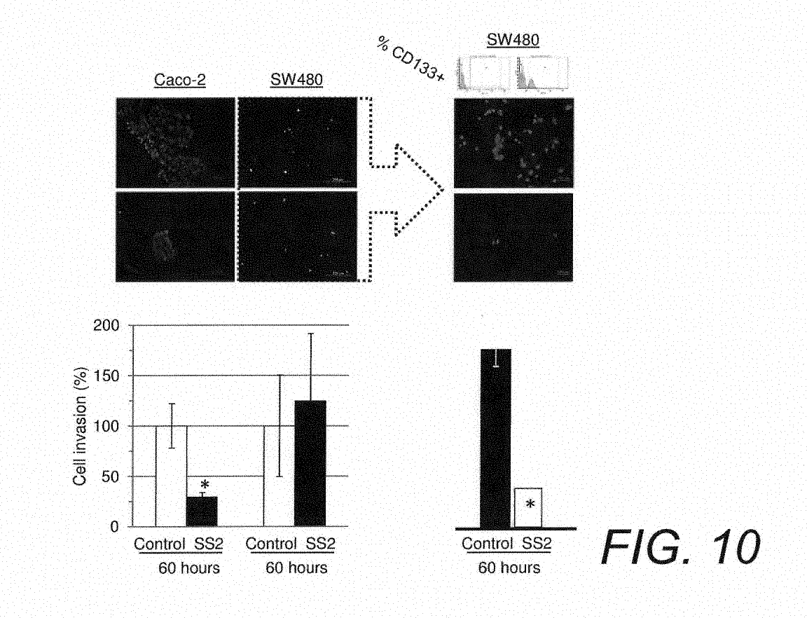

In the next step, we tested colorectal cancer cells for invasiveness after exposure to the SS2 virus. As expected, the Caco-2 cells with >70% CD133+ cells were much more inhibited in terms of their invasiveness, compared to SW480 cells with <4% CD133+ cells (FIG. 10). Interestingly, in another experiment when the level of CD133+ cells in SW480 cells was ˜34%, the invasiveness was inhibited in a significant manner (FIG. 10).

### Example 6

### Exposure to SS2 Induces Apoptosis

In many cases, oncolytic viruses destroy cancer cells by the way of necrosis through lytic infection. However, it has also been previously shown that exposure to oncolytic herpes can result in induction of apoptosis in non-permissive cells. Additionally, cells which are less permissive to oncolytic herpes seem to undergo apoptosis at higher levels. Similarly, induction of apoptosis was studied by evaluating the activation of caspase-3 in Caco-2 and SW480 cells after exposure to SS2. In order to evaluate apoptosis, an ApoLive-Glo™ multiplex assay (Promega, WI, USA) was used. Apoptosis was quantified by caspase-3/7 (a key biomarker of apoptosis) cleavage of a luminogenic substrate containing the tetrapeptide DEVD sequence (Asp-Glu-Val-Asp). Following such caspase-induced cleavage, a substrate for luciferase is released, thus resulting in the luciferase reaction and production of light.

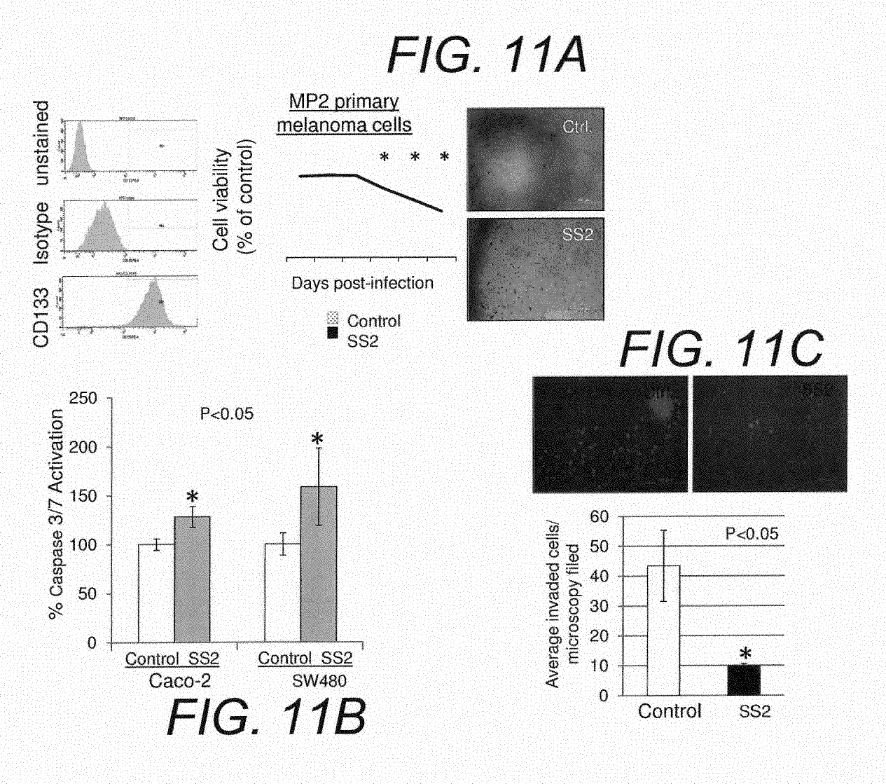

Both cells showed an increase in apoptosis 24 h post-infection, with SW480 cells, which showed lower permissiveness of SS2 as mentioned above, showing a higher level of caspase activation (FIG. 11B). Next, the outcome of exposure of primary cancer cells to SS2 was tested. Once primary MP2 melanoma cells were exposed to this virus, they lost viability starting at Day 3 post-infection. FIG. 11A shows the results of flow cytometry for CD133 in MP2 cells and cell viability post exposure to SS2 (MOI˜3). FIG. 11C shows loss of invasiveness for MP2 cells performed in 48 hours post-infection as previously explained by the data in FIGS. 9A-9C.

### Example 7

### SS2 Inhibits In Vivo Growth of Tumors in SC Mouse Model for Melanoma, HCC, and Colorectal Cancer

In order to evaluate the outcome of targeting CD133+ cells in vivo, we used subcutaneous (SC) xenografts of human cancer cells in male athymic nude mice (Hsd: Athymic Nude-Foxn1^{nu} 5-7 weeks of age, Harlan, Ind.) purchased from Harlan. In this model, 3-5×10⁶ cancer cells (UACC257, Caco-2, or Hep3B2) were injected in each flank to evaluate the potential growth inhibitory effects of SS2. Cancer cells were grown in vitro, trypsinized, re-suspended, and checked for viability using Trypan Blue/Giemsa staining prior to SC injection. The cells were either mixed with SS2 (MOI˜3) or with autoclaved 3% milk (vehicle of virus suspension as the control) immediately before SC injection into the mice. Cancer cells (3-5×10⁶) were injected subcutaneously in a 1:1 mixture of growth media/matrigel, with a final injection volume of 100 μl, to the flank of each subject bilaterally. The virus-treated cells were injected into the right flank, while the control-treated cells were injected into the left flank. Tumor growth was evaluated over the course of the next 4-10 weeks based on the cell line used. Tumor diameters were measured using a digital caliper on a weekly basis, and tumor volume was calculated by the formula [tumor volume (mm³)=(length [mm])×(width [mm])×0.52]. At the end point of the study, the animals were terminated and tumors were extracted, measured, and photographed.

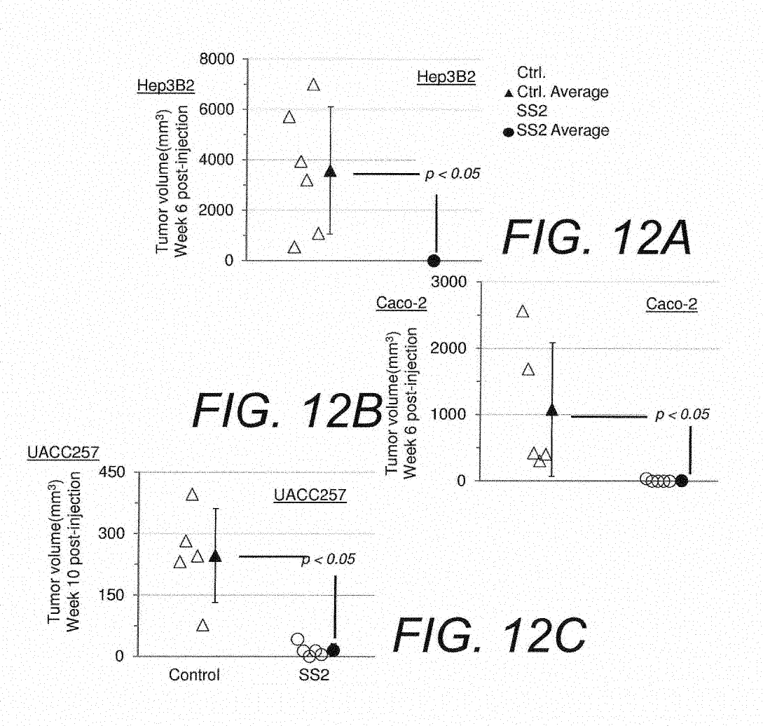

Individual tumor sizes and averages for each group are shown in the charts in FIGS. 12A-12C. In all three models investigated, a significant inhibition of in vivo growth was observed for the SS2 pre-treated side. Indeed, as shown in FIG. 12A, for the HCC model (Hep3B2 cells), there was no tumor growth in the test side, while the control-treated side developed SC tumors with an average size of ˜3600 mm³ at 6 weeks post-SC injection. In the colorectal cancer model (Caco-2 cells), the SS2 pre-treated side only developed one tumor with ˜37 mm³ volume, while the control-treated side developed five tumors with an average size of ˜1000 mm³ (FIG. 12B). At day 70 post-SC injection, melanoma cells UACC257 generated five tumors with an average size of ˜250 mm³ in the control side, compared with the average test side tumor size at ˜14 mm³ (FIG. 12C). Colorectal and melanoma tumors were extracted and photographed.

### Example 8

### SS2 Causes Regression in Established Melanoma Tumors

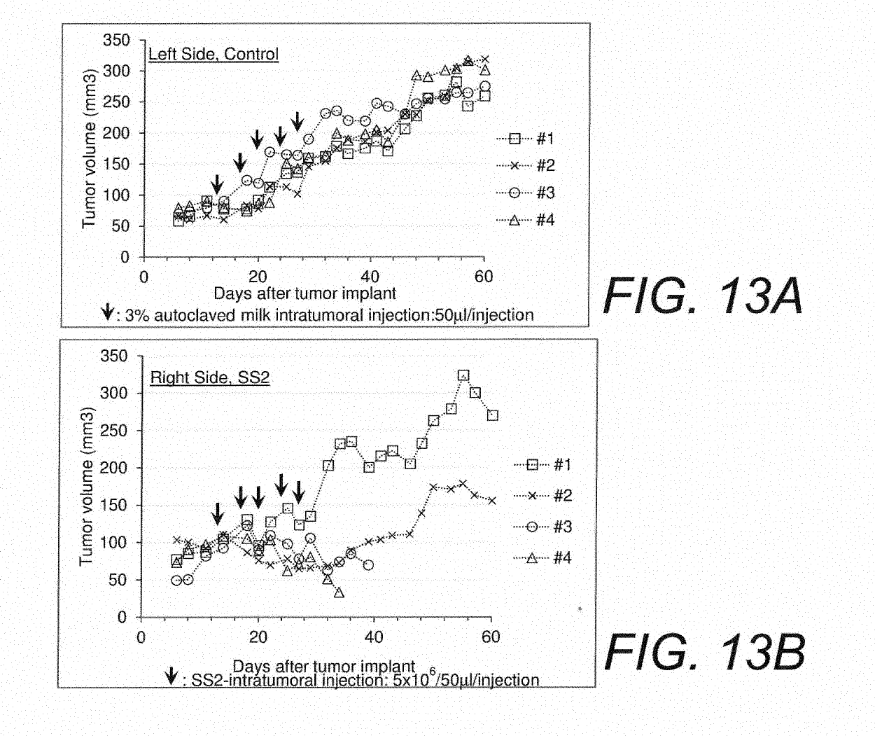

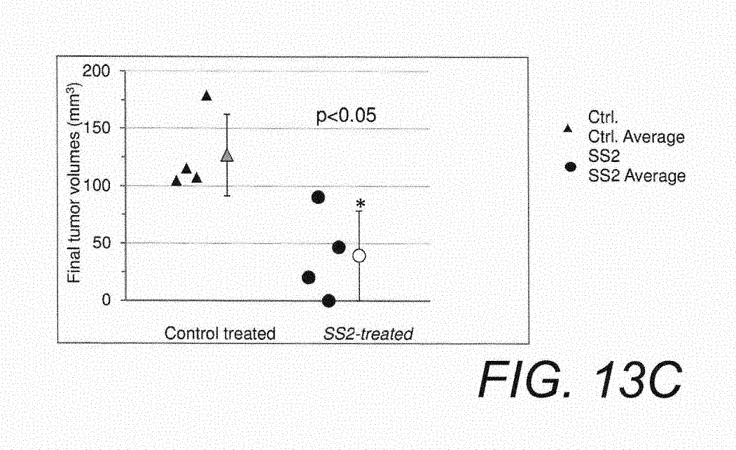

In this example, the potential of the SS2 virus to cause tumor regression in previously established tumors was studied in male athymic nude mice (Hsd: Athymic Nude-Foxn1^{nu} 5-7 weeks of age<Harlan, Ind.) were purchased from Harlan. For this model, melanoma UACC257 cells were used to establish SC tumors. 2.5×10⁶ cells were injected subcutaneously, and subsequent tumor development was followed by using a digital caliper. Once tumors reached 80-100 mm³, an injection of 5×10⁶ pfu/50 μl was administered to the tumors on the right side (test group), while the left side tumors (control group) were injected with 50 μl of 3% autoclaved milk. The injections on each side were repeated on a 3-day interval for a total of 5 injections for both sides in four subjects. Following this treatment, tumor growth was monitored for 60 days after the initial injection. At the conclusion of the 60-day period, all animals were sacrificed, and the tumors were excised, measured and photographed. FIGS. 13A and 13B show the course of tumor growth for both sides along with the treatment time points for all four mice. While three of the test-treated tumors regressed significantly upon injection of SS2 virus, all four control-treated tumors continued to grow to the end of the experiment. FIG. 13C shows the sizes of individual tumors at the last time-point. The average tumor size for the control group was ˜125 mm³ compared to 39 mm³ for the test group.

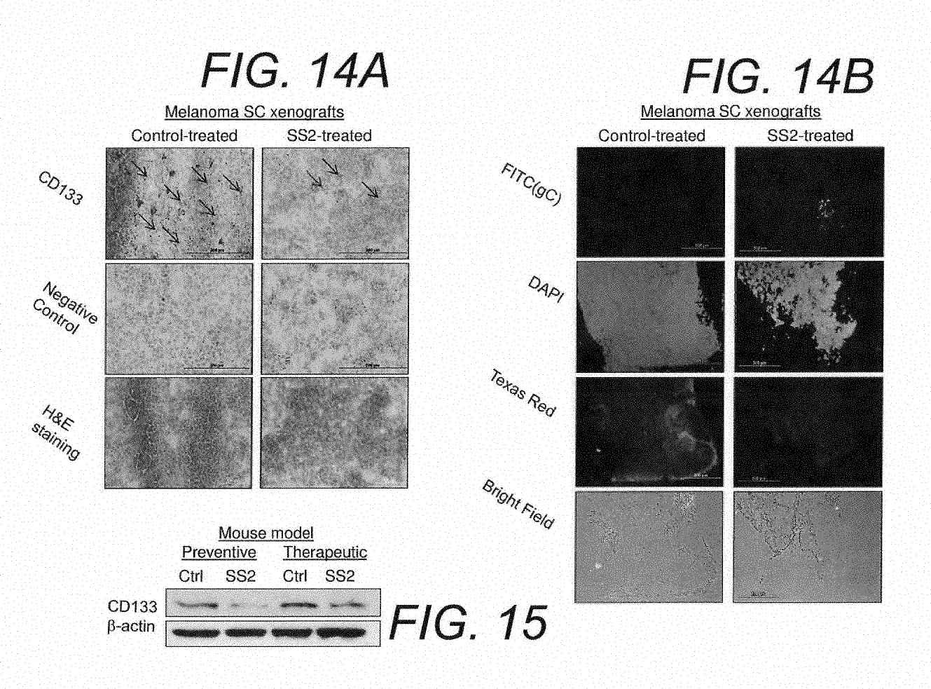

In the next step, it was important to investigate the replication of SS2 in treated tumors. Once extracted, tumors for this model were stained for CD133 expression. A significantly lower level of this marker (FIG. 14A, arrowheads point to brown staining for CD133) was observed in SS2-treated tumors compared to tumors injected with the vehicle. For histology studies, frozen sections were cut at 5-6 μm, adhered to Superfrost Plus slides, and air dried for 30 min. Slides were then fixed using room temperature acetone for 10 minutes and placed in tris-buffered saline (TBS). The primary antibody, CD133 at a 1:100 dilution (Miltenyi Biotec), was incubated at room temperature for 1 h, followed by TBS rinses. Detection was performed using Envision Plus anti-mouse labeled polymer and DAB Plus chromogen from Dako per the manufacturer's instructions. A light hematoxylin counterstain was applied, and the slides were dehydrated, cleared, and permanently mounted. Negative control slides were not treated with the antiCD133 (primary) antibody and showed no brown color staining. Tissues in this figure correspond to animal number 2 in FIGS. 13A-13B. FIG. 15 shows the expression of CD133 in lysates from tumor samples from preventive and therapeutic models. Blotting of these lysates revealed a decrease in both SS2-treated samples with a more robust reduction in the preventive model. Cell lysates were made using a single detergent lysis buffer [50 mM Tris (pH 8.0), 150 mM NaCl, 0.02% sodium azide, 100 mg/ml phenylmethy-sulfonyl fluoride, 1 mg/ml aprotinin, and 1% Triton X-100] normalized for the amount of total protein. The proteins (15-150 μg) were separated on 4-20% or 10% Criterion Precast Gel (BD Biosciences) and electro-transferred to nitrocellulose membrane. The membranes were then rinsed with distilled water and incubated with primary antibody overnight at 4° C., followed by incubation with secondary antibody. Protein bands were detected by an enhanced chemical luminescence kit (Millipore). Membranes were re-probed with β-actin or tubulin antibody to verify equal loading of proteins. In case of blotting for HSV-1 proteins, the membrane was then washed and incubated with a primary rabbit antibody against all HSV-1 antigens (Dako, CA, USA) for 2-8 h, followed by horseradish peroxidase (HRP)-conjugated secondary antibody for 2 h. After extensive washing, the membrane was exposed to Lumigel detection solution and subjected to autoradiography.

The replication of SS2 in treated tumors was also studied. As shown in FIG. 14B (first row, tissues represented in this Figure correspond to animal number 2 in FIGS. 13A-13B), expression of herpetic glycoprotein-C is observed in SS2-treated tumor tissue indicative of viral replication. The following rows show staining for DNA (DAPI), cytoplasm (Texas Red), and also light microscopy of the tissues.

### Example 9

### SS2 Causes Regression in Orthotopic Mouse HCC Model

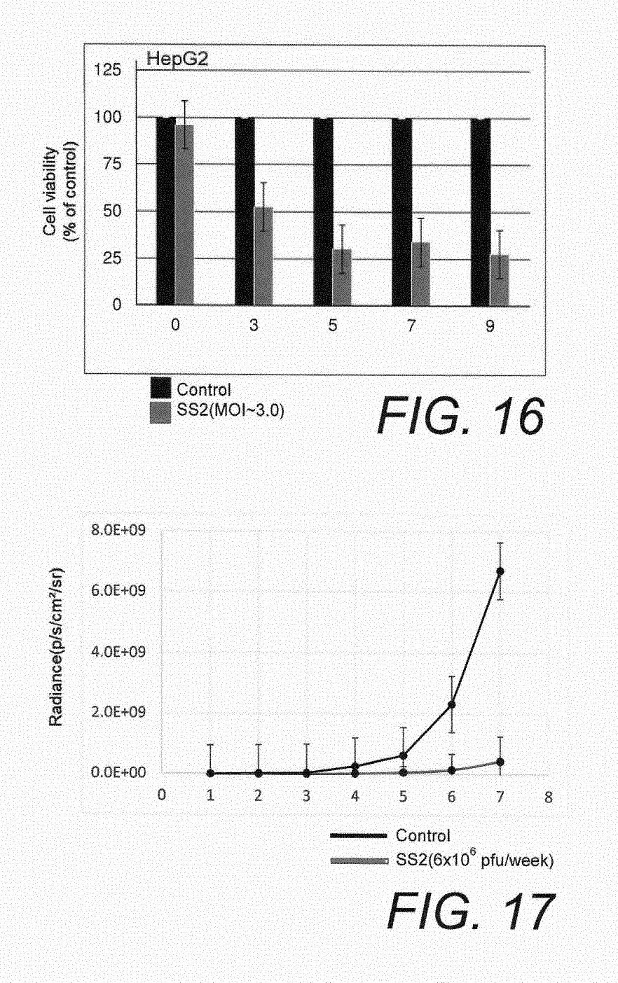

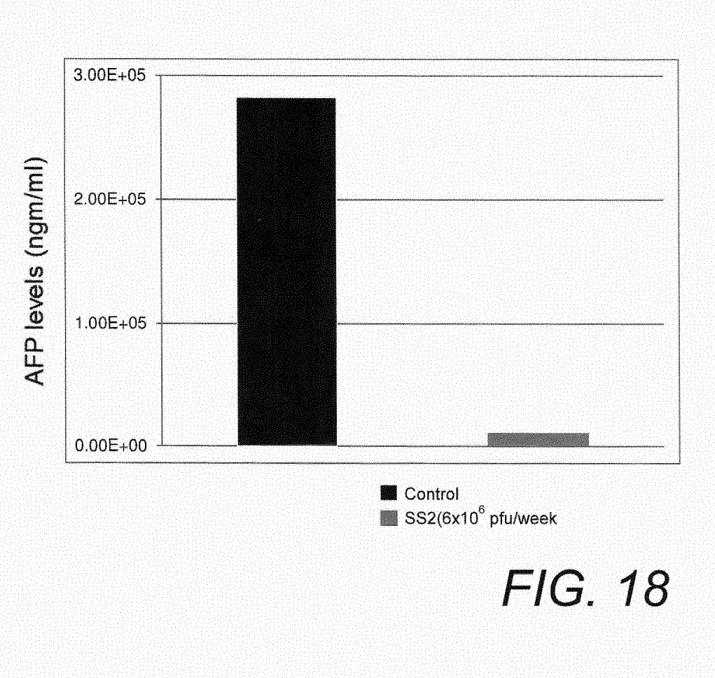

Systemic therapy using oncolytic viruses is a major clinical need because many tumor types are accessible mainly via systemic routes. In an effort to model systemic treatment using SS2, human-in-mouse orthotopic xenograft for HCC were used. An important feature of the cell used for this experiment, HepG2Luc, is the fact that it's only 4-6% positive for CD133. HepG2 cells were exposed to SS2 in-vitro (MOI˜3.0) for up to 9 days post-infection with a significant loss of viability observed as early as 3 days post-infection (FIG. 16). Next, intrahepatic liver tumors were generated by inoculation of 2.0×10⁵ HepG2Luc cells. An initial IVIS scan after a week was performed to evaluate the growth of liver tumors. The subjects underwent intravenous (IV) injections of SS2 (6×10⁶ pfu/week divided into 3 injections) or milk for 7 weeks. IVIS scanning was done on a regular basis, and those results are shown in FIG. 17. The average radiance (p/s/cm²/sr) of liver tumors from the control group was found to be close to 10-fold greater than the SS2-treated group. IVIS images before and after therapy showed significantly bigger tumors observed in control-treated animals than in SS2-treated animals. Finally, evaluation of human alpha fetoprotein (AFP, a marker for HCC) revealed 27-fold greater values in the control group in comparison to the SS2-treated group as an indicator of progression of the tumor (FIG. 18).

### Example 10

### SS2 does not Infect Vital Organs in Immune-Deficient Mice or Non-Malignant Human Kidney Cells

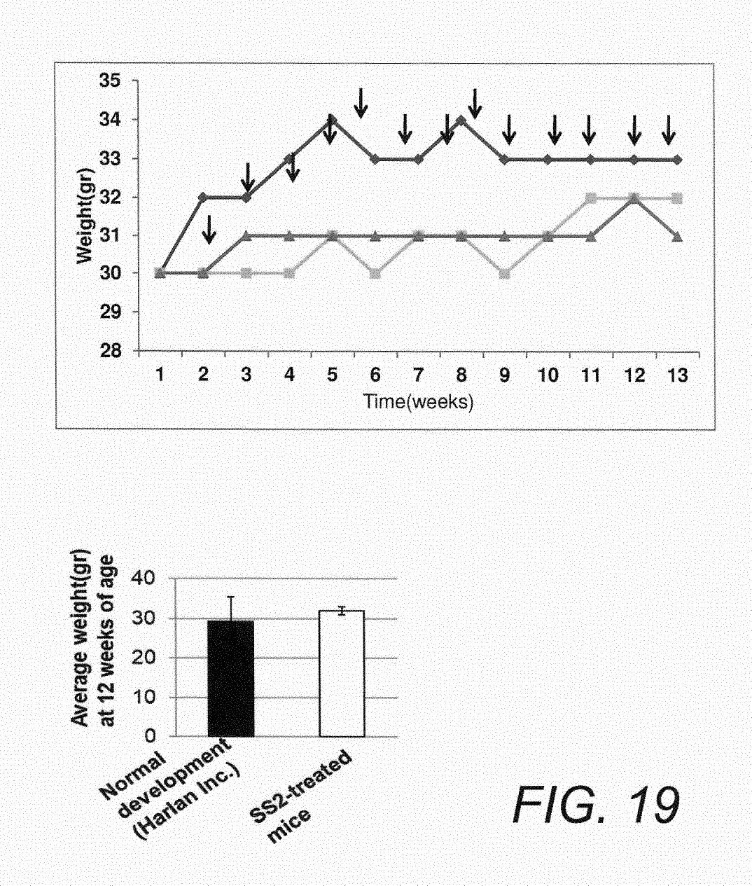

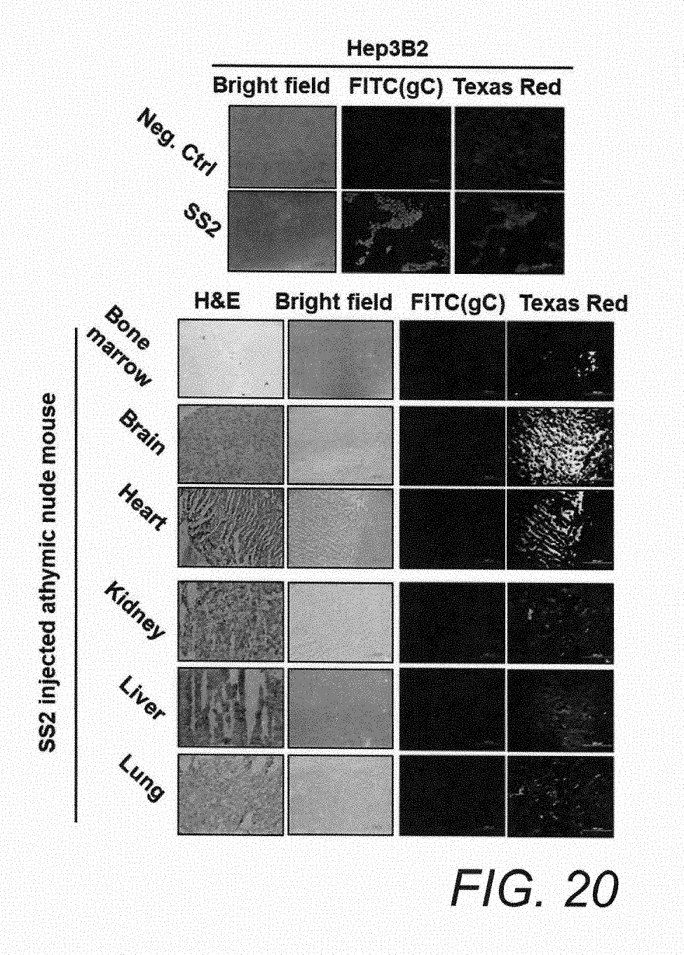

An important question for future use of SS2 virus is if this virus is capable of targeting vital organs and inducing toxic effects once injected systemically. To study this, the SS2 virus was injected into a cohort of three athymic nude mice (6-8 weeks of age) for 13 weeks with a weekly dose of 1.4×10⁷ pfu/week via IV and compared that with the regular course of animal growth. No signs of morbidity were observed, and the average weight of the animals was not affected by such therapy (FIG. 19). In the next phase, IF studies were performed using anti-gC on a range of vital tissues (i.e., bone marrow, brain, heart, kidney, liver, and lung tissues) extracted from SS2-treated animals in order to detect viral replication. All samples were negative for expression of gC, proving lack of replication of SS2 in vital organs (FIG. 20, lower panel). However, the positive control cells (Hep3B2) were infected and showed the expression of gC protein in this experiment (FIG. 20, upper panel). Additionally, the viability of normal human kidney cells (NHK), known for expression of CD133, was not influenced with exposure to the SS2 virus. No statistically different changes are seen in the viability of NHK cells exposed to SS2 (MOI˜3) versus control (3% autoclaved milk)-treated cells.

### Example 11

### Cell Culture

E5 and Vero cells (American Type Culture Collection, ATCC, VA), human fetal astrocytes (Lonza, MD) and glioma cell lines (ATCC) were grown in Dulbecco's Modified Eagle's Medium (DMEM) (Sigma-Aldrich, MO) with 10% fetal bovine serum (Thermo Fisher Scientific, MA) and 1% penicillin and streptomycin (Sigma-Aldrich) at 37° C., 5% CO₂ and 95% relative humidity. NCCIT cells (ATCC) were grown in Roswell Park Memorial Institute (RPMI)-1640 media with 10% FBS and antibiotics under similar incubation conditions. U87 expressing luciferase (U87MGLuc) were obtained from Perkin Elmer (OH) and grown in DMEM 10% FBS. Cells were passaged at 70-80% confluency

### Example 12

### Construction of Plasmid Containing ICP4 Gene Controlled by Ezh2 Promoter

A 1.1 kb fragment (1,143 bps, sequence spanning −1,095 to +48)(Poulsen et al., 2008) of the human EZH2 promoter was amplified by nested PCR from human genomic DNA purified from cultured NCCIT cell (ATCC: CRL-2073). First round PCR primers were: sense 5′-tgcagaggcggcaagtgaacag-3′ and antisense ttctgagtcccaccgggtgtcg-3′. Second round PCR primers were: 5′-taccggtgaactacgaacagtgg-3′ (sense, AgeI site was created, underlined letters) and 5′-tactagtcggaccgagcgccaaac-3′ (antisense, SpeI site was created, underlined letters). The PCR fragment was inserted into pTIIDT (Pan et. al, 2009, as described above in Example 1) between the HSV-1 thymidine kinase (TK)-5′ DNA and ICP4 gene via AgeI and SpeI restriction sites. The resulted plasmid was named as pTEZH2IIDT (FIG. 21A).

### Example 13

### Generation and Purification of SS3 Virus

E5 cells were transfected with pTEZH2IIDT using Lipofectamine 2000 (Thermo Fisher Scientific; Invitrogen, CA) according to the manufacturer's instructions. Cells were infected with d120 virus 72 hours later, thereby creating a recombinant herpes virus expressing the ICP4 gene under control of the human EZH2 gene promoter. The virus was harvested 3 days post infection. Vero cells were used for recombinant virus purification. The virus was passed in Vero cells for three rounds to eliminate ICP4-negative variants. The last batch of virus was then serially diluted (10⁻¹ to 10⁻), A volume of 500 μL from each dilution was added to duplicate wells in a 24-well plate (BD Biosciences, CA) containing Vero cells (˜80% confluency) after removal of existing media. The cell culture was harvested 4 days later. The well with the highest dilution and positive virus signal by HSV polymerase was considered to contain the isolated virus (10⁻⁶). Viral titration was performed through immunofluorescence microscopy. The virus was stored in autoclaved 3%-nonfat milk at −80° C. with various concentrations between 1-6×10⁴ particles per microliter (μL).

The genomic composition of this recombinant virus was identified and confirmed using PCR by different primer pairs (FIG. 21B). Independent viral isolates were investigated with the purpose of confirming the structure of the recombinant virus. This resulting virus was named the Signal-Smart 3 (SS3) virus. The structure of the recombinant promoter was confirmed by performing PCR. In each case, the PCR product was sequenced and compared to the related sequence in our database. The HSV-1 polymerase, Kanamycin gene, TK/EZH2 promoter, DsRedExpress, and ICP4/IRES/DeRedExpress/TK were analyzed and confirmed by PCR (FIG. 21B). The second row data (Kanamycin gene) is used as the evidence that the viral DNA preparation is not contaminated with pTEZH2IIDT. Restriction maps of the aforementioned genomes and the genome of d120 were produced by BamH1 digestion (Brown and MacLean, 1998). Viral DNA was purified by a Qiagen DNeasy Blood & Tissue Kit (Valencia, Calif.) according the manufacturer's instructions.

### Example 14

### Restriction Analysis of Viral DNA

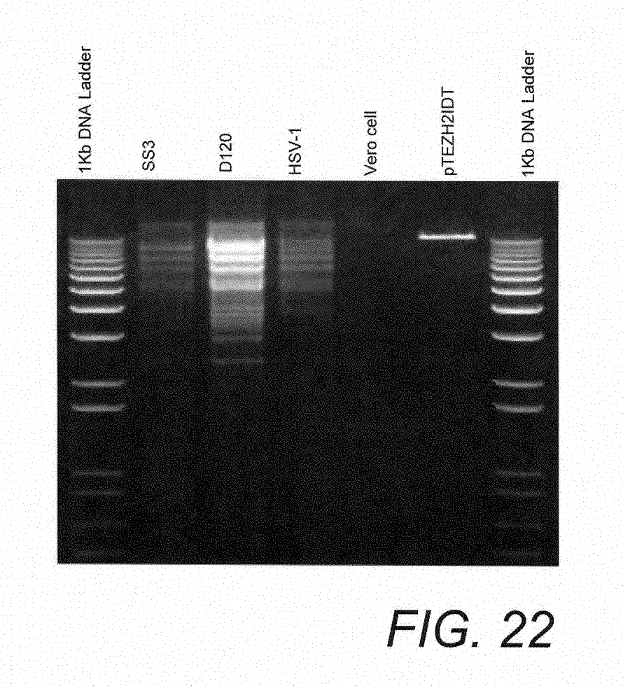

Viral DNA was purified as previously described in (Pan et al., 2009). E5 cells were grown up in a 10 cm culture dish and infected with the SS3 virus, and the infected cells were harvested when maximum cytopathic effects (CPE) were observed. Cells were then lysed by three freeze-thaw cycles with a dry ice ethanol bath. After spinning at 5000 rpm 4° C. for 10 minutes, the supernatant was treated with RNase A and DNase I at 37° C. for 2 hours, and subsequently, it was treated with protease K and Sodium dodecyl sulfate (SDS) at 56° C. for 1 hour. Viral DNA was then purified by a Qiagen DNeasy Blood & Tissue Kit (Cat. No. 69506) according to manufacturers' instructions. Purified viral DNA was digested with restriction enzyme BamH1 and analyzed in 1% agarose gel. Pattern similarities in restriction maps between SS3, d120, and parental HSV-1 indicated that there were no major differences in overall genomic structure between the viruses (FIG. 22).

### Example 15

### Ezh2 is Highly Expressed in GBM Cells but not in Non-Malignant Human Astrocytes

In order to confirm the increased expression of Ezh2 in GBM, five well-studied and widely-used glioma cell lines were examined along with fetal human astrocytes (feHA). Western blotting showed high levels of protein across all cancerous cell lines but not in feHA (FIG. 23). Cells were washed with phosphate buffered saline (PBS) (Sigma-Aldrich) and lysates were collected with single detergent lysis buffer (Cell Signaling, MA). Protein quantification was performed with the Coomassie Protein Assay Kit (Thermo Fisher Scientific) and read on a Benchmark Plus microplate spectrophotometer (Bio-Rad, CA) using Microplate Manager software (Bio-Rad) to analyze and interpret the absorbance values. Total protein concentration was normalized between samples. SDS-PAGE electrophoresis was performed using 10% Criterion Precast Gel (BD Biosciences) and electro-transferred to nitrocellulose membrane. Membranes were incubated overnight at 4° C. with gentle agitation in Ezh2 primary antibody (Cell Signaling) and then incubated at room temperature in secondary antibody conjugated to horseradish peroxidase (HRP). Protein was detected by an enhanced chemical luminescence kit (Millipore, MA). The same detection was done for β-actin using primary antibody conjugated with HRP (Cell Signaling) to confirm equal loading of protein. Primary and secondary antibodies were purchased from Cell Signaling other than anti-HSV-1 polyclonal antibody which was purchased from Dako (CA).

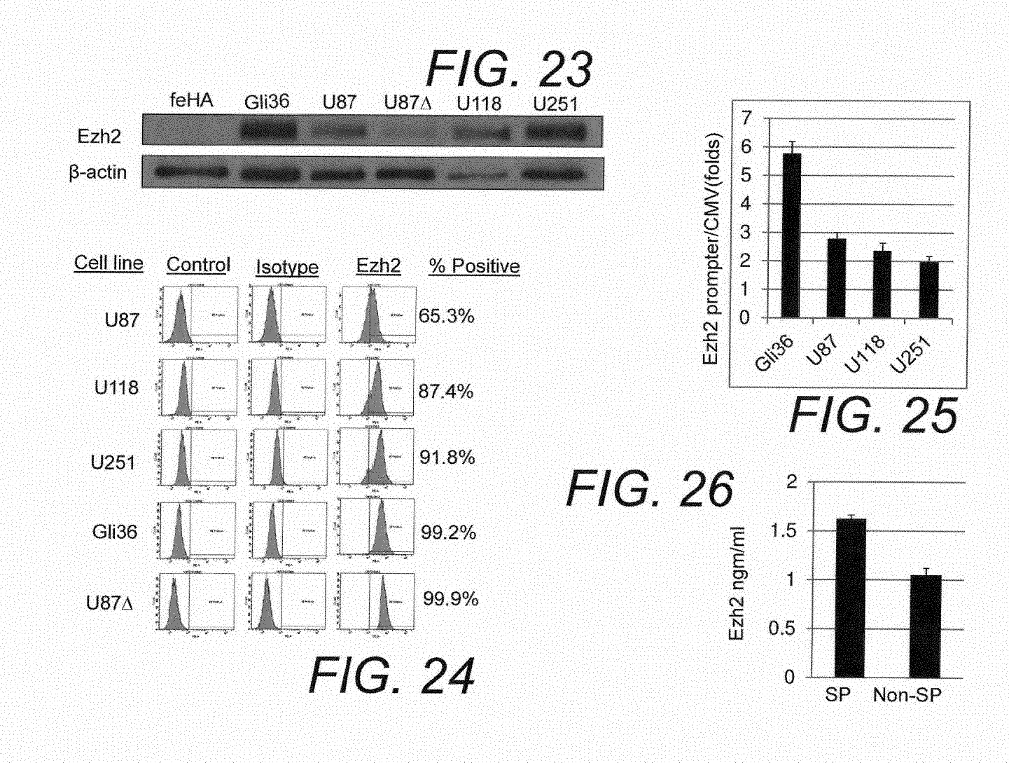

Further examination by flow cytometry showed that in cancer cell lines, the proportion of cells expressing Ezh2 ranged from 65.3% to 99.9% (FIG. 24). Cells were prepared using a Cytofix/Cytoperm kit (BD Biosciences). Cells were fixed by incubation with Cytofix/Cytoperm solution on ice for 30 minutes. They were then incubated with Ezh2 primary antibody for 30 minutes. Cells were then washed with Perm/Wash buffer three times before incubation with phycoerythrin (PE)-conjugated secondary antibody (Cell Signaling, MA). The cells were then washed three more times and re-suspended in wash buffer. Isotype samples were incubated with PE-conjugated secondary antibody only and controls were not incubated with any antibodies. Flow analysis was performed with a BD LSR II flow cytometer (BD Biosciences).

Further, Ezh2 promoter activation was analyzed using a luciferase reporter assay. FIG. 25 shows that Ezh2 promoter was active in GBM cells ranging from 2 to 5.8 folds as compared to the CMV promoter as control. Dual-Luciferase® Reporter Assay System (Promega, WI) was used for the promoter assay. The Ezh2 promoter (1.1 kb) was cloned into reporter vector pGL4.10 directly upstream of the firefly luciferase gene. The pGL4.75 plasmid, expressing the Renilla luciferase using cytomegalovirus (CMV) promoter, was used as internal normalization control. The assay was performed following the manufacturer's instructions (Promega). Briefly, 1 μg experimental plasmid and 1 ng control plasmid were co-transfected into 1-2×10⁵ cells, which were cultured on a 24-well plate, using Lipofectamine® 2000 reagent (Lifetechnologies, CA). Cell lysates were prepared 24 hours post transfection using Passive Lysis Buffer (Promega). Cell debris was removed via centrifugation and Luciferase Assay Reagent was added to the supernatant. Bioluminescence was measured and compared using a GloMax illuminometer (Promega).

### Example 16

### Ezh2 Expression is Higher in Side Population (SP) of Glioma Cells as Compared with Non-Side Population Cells (Non-SP)

SP cells are a sub-population of cells that are different from the main population on the basis of characteristics used for their isolation. In this study, the efflux capacity of cells was used against DCV in order to sort cells with high efflux (SP cells) that are enriched in glioma stem cells (GSCs). Cells with low efflux are considered to be of differentiated nature and are referred to as non-SP cells. Glioma stem cells (GSCs) were sorted with a modified version of the Hoechst side population (SP) analysis, using Vybrant® DyeCycle™ Violet (DCV) (Thermo Fisher Scientific) as described previously (Mathew et al., 2009; Telford et al., 2007). U251 glioma cells were passaged at 80-90% confluency from 100 cm cell culture dishes and suspended in DMEM for 15 minutes at 37° C. Verapamil (Sigma-Aldrich) 50 μM was added to the efflux pump inhibitor control group and incubated for 15 minutes. Afterwards, DCV 10 μM was added to all groups and incubated for 90 minutes at 37° C. with frequent agitation. Cells were then pelleted by centrifugation and re-suspended in an ice-cold SP buffer (Hank's Balanced Salt Solution [HBSS] containing 2% FBS and 2 mM 4-[2-hydroxyethyl]-1-piperazineethanesulfonic acid [HEPES] buffer). 7-amino-actinomycinD (7-AAD) (Thermo Fisher Scientific) was added for viability assessment. SP cells were sorted using a BD FACSAria cell sorter (BD Biosciences). Both SP and non-SP cells were lysed for enzyme-linked immunosorbent assay (ELISA).

Once analyzed in U251 cells, isolated SP cells examined by ELISA expressed 55% higher levels of Ezh2 (1.63±0.04 ng/ml) as compared with non-SP cells (1.05±0.07 ng/ml) (p=0.009, FIG. 26). This has been interpreted as a level of evidence that Ezh2 may exert a role in the biology of GSCs. ELISA for Ezh2 was performed using a “sandwich” style kit (Cloud Clone Corp., TX). Standards and samples in triplicate were incubated for 2 hours in a 96 well plate that had been pre-coated with Ezh2 antibody. After incubation, Ezh2 antibody conjugated with biotin is added and incubated for one hour. Avidin conjugated with HRP is then added and incubated for 30 minutes. Incubations with these detection reagents were followed by three to five washes with a mild detergent wash buffer. The substrate solution was then added and incubated for 15 minutes before reaction termination with sulfuric acid. The plate was read by spectrophotometer at 450 nM.

### Example 17

### SS3 Virus Targets GBM Cells and Reduces their Proliferation and Invasiveness

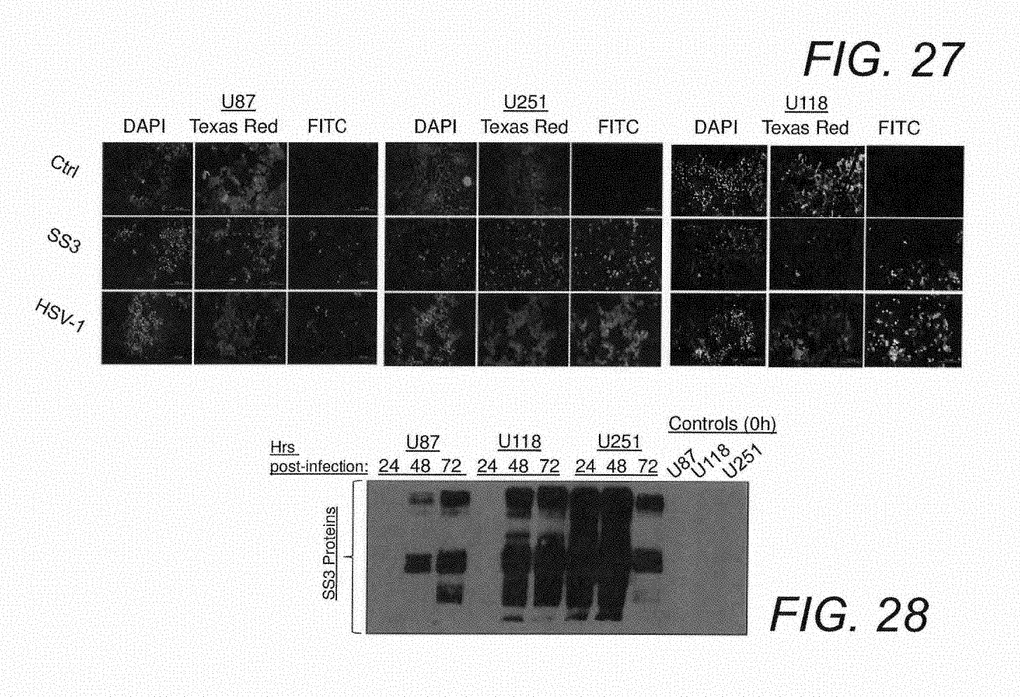

The expression of HSV-1 envelope protein glycoprotein-C (gC) is used as an established marker for replication of the virus. Three GBM cell lines were analyzed with immunohistochemistry (IHC) for expression of gC (green fluorescence, FITC) after being exposed to the SS3 virus (MOI˜3) or a control (3% autoclaved nonfat-milk as the vehicle for SS3) for 36 hours. As displayed in FIG. 27, all cancer cells studied (U87, U118 and U251) are permissive to SS3 and HSV-1. Blue staining (DAPI) shows nuclei of all cells, while red staining (Texas Red) shows cytoplasm. Up to 10,000 cells were seeded in 8-well slide chambers (Falcon, Calif.) and allowed to incubate overnight. The next day, designated wells were used for cell counting, and two wells each were infected with SS3 or HSV-1 at an MOI based on the proliferation assay. Control wells were given 3% autoclaved nonfat milk as the vehicle for the virus. At different times post-infection, again dependent on proliferation assay results, cells were fixed in acetone for 15 minutes and allowed to dry at room temperature. The wells were then incubated with a fluorescein-labeled mouse monoclonal antibody against HSV-1 glycoprotein C (gC) antigen and Texas Red stain (Labvision, MI) for 30 minutes at 37° C. The slides were then washed, dried, and mounted using UltraCruz Mounting Medium containing DAPI stain. Images were acquired with an inverted Olympus DP71 microscope and digital camera.

The profile of SS3 viral proteins produced in GBM cell lines is shown in FIG. 28 by using a polyclonal antibody raised against HSV-1 antigens in a western blot experiment. All three GBM cell lines produced SS3 proteins, while non-infected controls are void of viral proteins.

Next, the outcome of SS3 replication on viability and invasiveness of cancer cells was evaluated. FIG. 29 displays proliferation assays showing a significant reduction starting at 72-96 hours post-infection. One thousand cells were plated onto 96 well plates (BD Biosciences) and incubated overnight in 100 μl of DMEM. The next day, designated wells were trypsinized and cells were counted. Wells of interest were infected starting at a multiplicity of infection of 3 (MOI˜3). Each 24 hours, up to 144 hours, 49 μl of XTT reagent (Cell Signaling) and 1 μl of electron coupling solution were added to three control and three infected wells and incubated for one hour. Colorimetric viability was assessed by spectrophotometer at 450 nM.

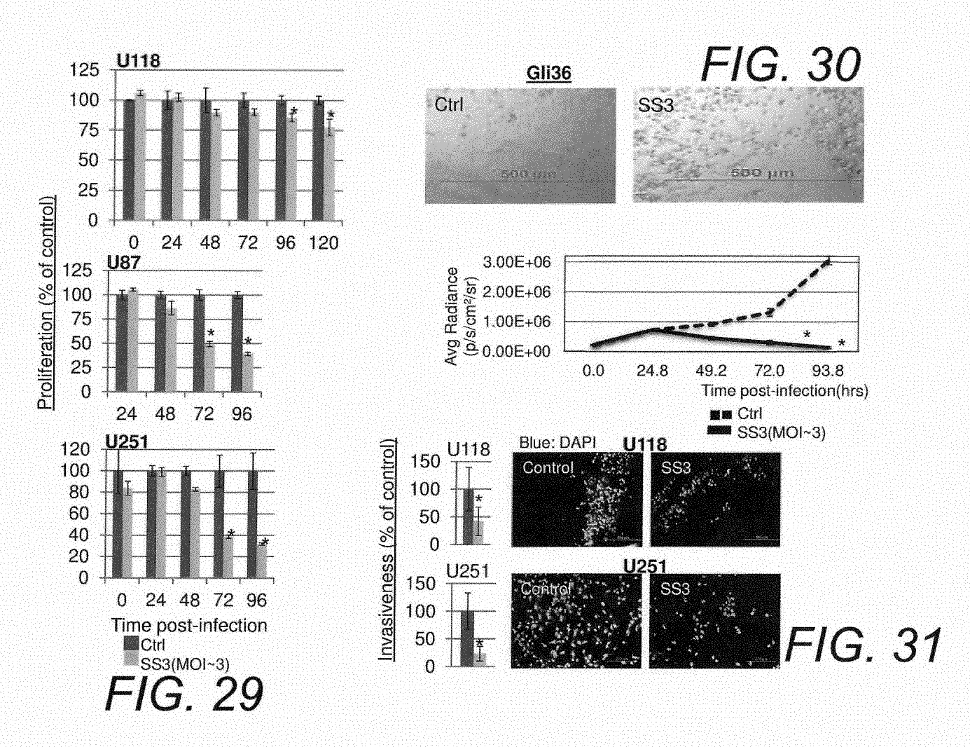

Classical signs of infection such as rounding and clumping were observed as early as 2-3 days post-infection for Gli36 cells, as shown in FIG. 30. Additionally, a luciferase expressing version of U87 cells named U87MG Luciferase (U87Luc) was treated with MOI˜3 of the SS3 virus. A significant reduction in radiance (p/s/cm²/sr) of U87Luc cells occurred for SS3-treated cells but not for control-treated cells once luciferin was added to the media and cells were imaged by the IVIS system. FIG. 30 further shows quantification of the radiance of control-treated U87Luc cells as compared with SS3-treated cells as a consequence of cell death by SS3.

Additionally, cell invasion (evaluated by a modified Boyden chamber assay) was severely inhibited at early time post-infection (20 hours) when viability of cells is not affected. Assays of U118 and U251 showed reduction of invasiveness to 42.3±25.7 (p=0.05) and 22.9±13.3 (p=0.001) percent of control, respectively (FIG. 31). Blue staining (DAPI) shows nuclei of invaded cells. A modified Boyden chamber assay was performed using a BioCoat Matrigel Invasion Chamber (BD Biosciences). Two chambers were prepared for each cell line by placement in 24-well plates and incubating in DMEM for two hours. Afterwards, 25,000 cells were seeded into the chamber and incubated up to four hours until cell attachment to the Matrigel membrane. The SS3 virus was then added to the chamber of interest, up to an MOI as determined by the cell line's proliferation assay. The membrane was then washed, dried, and fixed with methanol up to 36 hours after viral seeding. Cells on the inside attachment surface of the membrane were removed by gentle rubbing with a cotton swab prior to fixation. Cells that invaded to the other side of the membrane were detected by DAPI (4′-6-Diamidino-2-phenylindole) staining with UltraCruz Mounting Medium (Santa Cruz Biotech, CA).

### Example 18

### SS3 Virus Inhibits the Growth of Mouse Glioma Cells but not Non-Malignant Mouse Astrocytes

The RCAS/TVA (replication competent ALV-LTR, splice receptor/Tumor Virus A) system has been successfully utilized to model gliomas. In this model, the TVA receptor is expressed from the nestin promoter (N-TVA) restricting its expression to neural and glial stem and progenitor cells. Mutant Kras (G12D) expression combined with either Akt activation or deletion of the Ink4a/Arf locus in N-TVA; Ink4a/Arf^{lox/lox} mice results in the generation of high-grade gliomas. To evaluate the effects of SS3 on mouse glioma cells, three different cell types were tested: 1-Kras^{G12D}/Akt expression along with Ink4a/Arf deletion, named as MC41-Kras/Akt/Cre glioma cells; 2-EGFRvIII/Akt expression along with Ink4a/Arf deletion, named as EGFRvIII/Akt/Cre glioma cells; and 3-Ink4a/Arf deletion, named as MC41-Cre cells, which are considered non-transformed background cells.

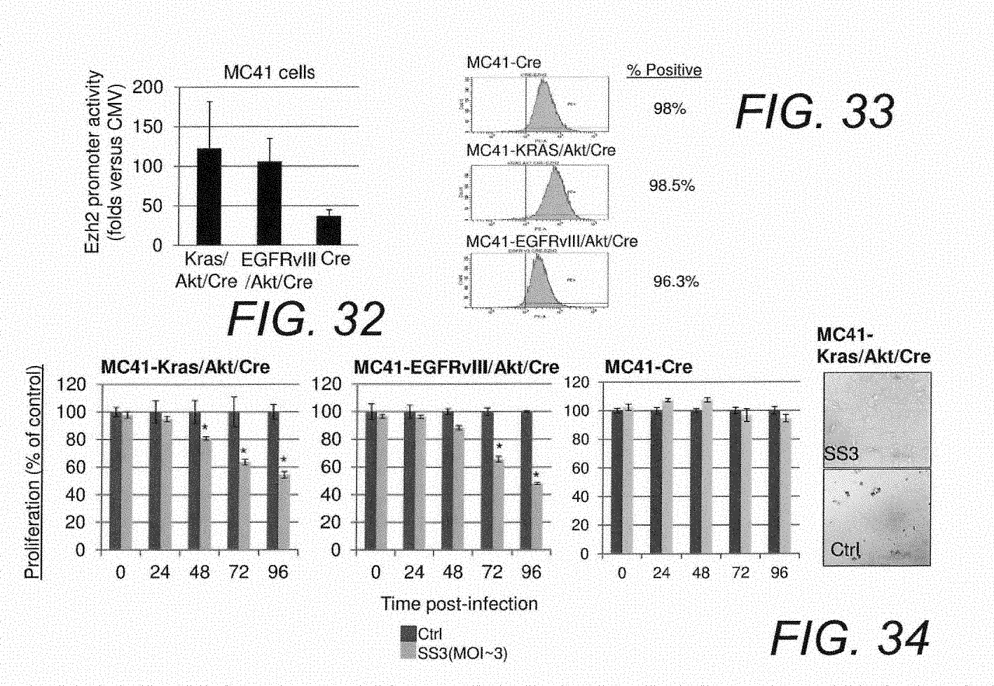

Since the SS3 virus is constructed with the human Ezh2 promoter, it was important to determine if this promoter is active in mouse glioma cells. This was tested by transfecting cells with a reporter construct expressing luciferase from the human Ezh2 promoter in comparison to expression of luciferase from the cytomegalovirus (CMV) promoter as a constitutive promoter. As shown in FIG. 32, MC41-Kras/Akt/Cre and MC41-EGFRvIII/Akt/Cre mouse glioma cells contain higher levels of Ezh2 promoter activation in comparison with MC41-Cre cells.

Flow cytometry data showed that all three cell lines contained Ezh2+ cells (FIG. 33). Exposure to SS3 at MOI˜3 resulted in significant loss of viability in glioma cells but not in the background MC41-Cre cells due to lower promoter activity in these cells (FIG. 34). FIG. 34 further shows the morphology of infected MC41-Kras/Akt/Cre cells at 72 hours post-infection.

### Example 19

### SS3 Infection Prevents Tumor Development in Subcutaneous (SC) Mouse Model

The SS3 virus' ability to combat the growth of U87 cells in-vivo was studied in a preventative mouse model. For this purpose, 1.5×10⁶ U87 cells (1:1 media/matrigel) were mixed with the SS3 virus (MOI˜3) or a control (3% autoclaved nonfat-milk as the vehicle for SS3) and injected via SC in the flank area of an athymic male nude mouse (Hsd: Athymic Nude-Foxn1^{nu}, Harlan, Ind.) at 6-8 weeks of age.

Tumor cell cultures were passaged, pelleted, and re-suspended in a 1:1 mixture of DMEM/Matrigel (BD Biosciences) and MOI˜1 of SS3 virus or an equivalent volume of 3% autoclaved nonfat-milk. Two million cells were subcutaneously injected in each flank of the mice, with the left side serving as control and the right side receiving cells that were mixed with SS3. Tumor sizes were measured with digital calipers twice weekly for four weeks. Tumor volume was estimated by the formula: Tumor Volume (mm³)=length×width²*0.5. After four weeks, the mice were sacrificed, and the tumors were extracted and re-measured.

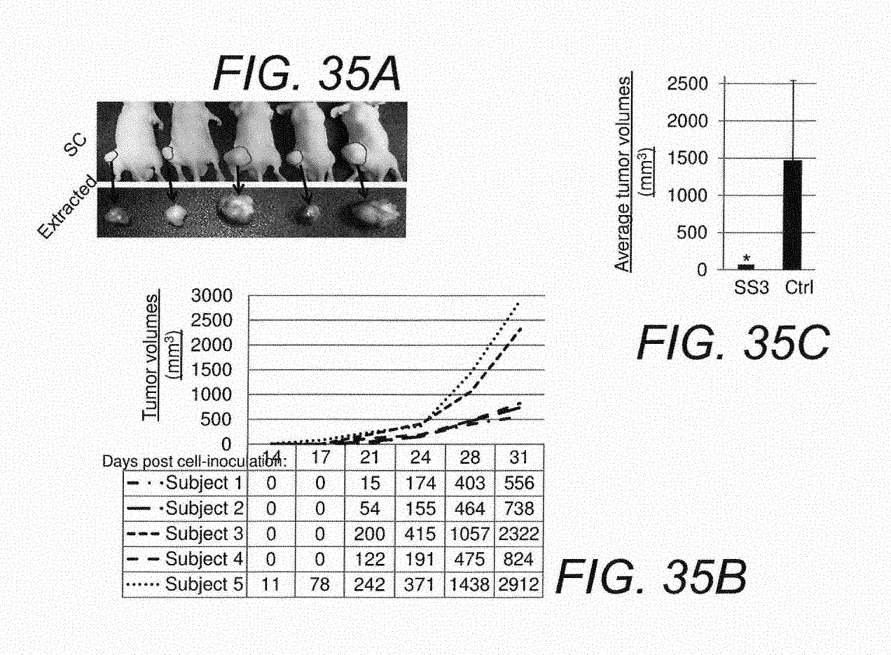

Tumor engraftment and growth in all animal subjects were completely inhibited in the right flank (SS3-treated), while the left flank (control-treated) gave rise to sizeable tumors during 31 days after SC cell inoculation. FIG. 35A shows SC tumors and extracted tumors at last time point (day 31). FIG. 35B shows the course of tumor growth for each subject and related volumes. Average tumor sizes for SS3 and control-treated groups are shown in FIG. 35C reaching ˜1400 mm³ in control groups while no growth was observed in the SS3-treated groups.

### Example 20

### SS3 Infection Inhibits the Growth of Pre-Established SC Human Tumors in Nude Mice

To test the SS3 virus for inhibition of pre-established tumors, 2.0×10⁶ U87Luc cells were suspended in a 1:1 mixture of DMEM/Matrigel and injected via SC in the flank area of male athymic nude mice. Tumors were measured twice weekly, and the tumors were allowed to grow to ˜80-100 mm³. At this volume, the SC tumors were injected with 6×10⁶ plaque forming unit (pfu) of the SS3 virus (in 100 ml of 3% autoclaved nonfat-milk) on one side while the tumor on the other side was injected with nonfat milk upon reaching the same volume. All tumors received an injection of the SS3 or milk on a weekly basis up to 6 injections, and the tumor volume was measured up to 6-10 weeks post initial cell inoculation. The mice were sacrificed when the tumor burden and disease process impacted quality of life or reached >250 mm³ as determined by IACUC protocol. Tumors were extracted, re-measured, and graphed for their growth curve and also averages. The radiance of SC tumors was evaluated by injecting 0.3 ml of 15 mg/ml of luciferin solution 10 minutes ahead of IVIS imaging. The luminescence of U87Luc cells (represented by region of interest or ROI) was detected using IVIS® (IVIS SpectrumCT Pre-clinical In Vivo Imaging System) following injection of the luciferin solution and graphed as photons/s⁻¹cm⁻²sr⁻¹.

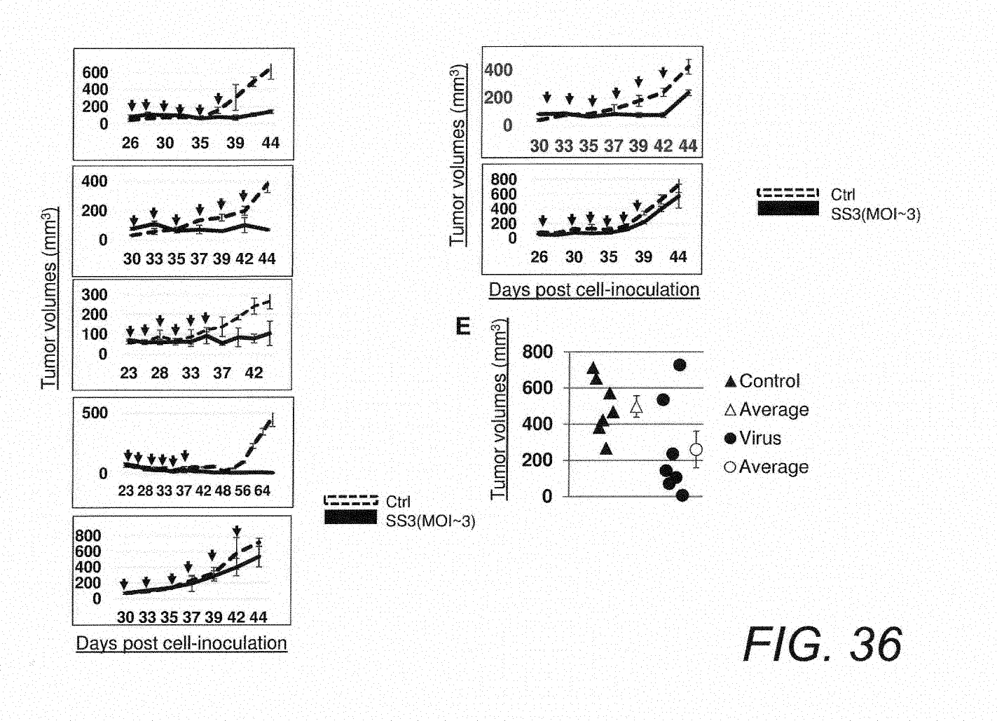

A shown in FIG. 36, significant tumor regression or delay in progression was observed as evidenced by tumor volume measurements in SS3-treated groups in comparison with control-treated groups. Imaging using an IVIS system for radiance of U87Luc cells also showed tumor regression or delay in progression for SS3-treated groups in comparison with control-treated groups. Average tumor volumes were ˜500 mm³ for control-treated tumors and 260 mm³ for SS3-treated tumors by the end of treatment course.

### Example 21

### Single Injection of SS3 Inhibits Intracranial Tumor Progression

Intracranial injection of U87Luc (3×10⁵/6 μL injection) cells were used to establish orthotopic tumors in the brain of athymic nude mice approximately 6 weeks of age. For each mouse, after proper anesthesia, a 1 cm longitudinal incision was made. The bregma was identified, and a small burr hole was made 2 mm lateral and 1 mm posterior. Using a Kopf stereotactic frame, 6 microliters of the U87 cells solution was carefully injected 2.5 mm deep into the brain. The burr hole site was judiciously observed to make sure that fluid was not refluxing out of the syringe. After completion of the injection, the syringe was left in place for 1 minute prior to withdrawal. The mouse was removed from the stereotactic frame and veterinary tissue glue was used to close the incision. The mouse was then placed on a heating pad until the animal woke up.

At two weeks post cell inoculation and confirmation of tumor growth by IVIS, the mice received a single injection of SS3 (2.0×10⁵ pfu/20 μL) or control (milk) at the approximate same location of original intracranial tumor cell inoculation. Each mouse was sufficiently anesthetized using isoflurane, first in an induction chamber and then attached to a mouse gas anesthesia head holder through which the mouse continued to receive isoflurane. The previous incision was reopened, and the burr hole was visualized. Either virus or milk was aspirated into a Hamilton syringe, which was then injected through the burr hole using the stereotactic frame. 20 microliters of SS3 or control was slowly injected 2.5 mm deep into the brain. The incision was then reclosed using veterinary tissue glue. The mouse was removed from the head holder and placed on a heating pad and monitored until recovery from anesthesia.

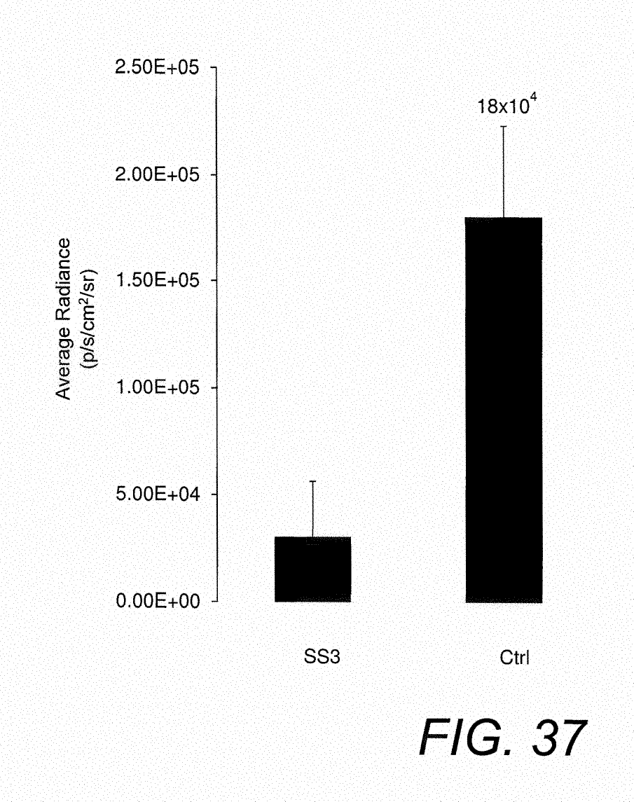

Intracranial tumors were imaged using IVIS at 1 week post treatment. A reduction in the radiance of intracranial tumors was observed once tumors before and after therapy were compared. FIG. 37 shows the average of radiance for SS3-treated subjects and control-treated subjects, with the SS3-treated group radiance averages being ˜16% of the control group.

Having thus described the invention, what is claimed as new and desired to be secured by Letters Patent is:

1. A recombinant oncolytic virus configured for transcriptionally targeting cells expressing a pro-oncogenic factor, wherein:
a promoter for a viral gene controlling viral replication is deleted;
a natural or synthetic sequence capable of driving expression of said viral gene controlling viral replication and including a promoter for said pro-oncogenic factor is inserted in controlling relation to said viral gene controlling viral replication in place of said promoter for said viral gene controlling viral replication such that said promoter for said malignant factor controls expression of said viral gene controlling viral replication;
said viral gene controlling viral replication is only expressed within cells expressing said pro-oncogenic factor; and
expression of said viral gene controlling viral replication results in replication of said virus within and destruction of an infected cell.

2. The recombinant oncolytic virus according to claim 1, wherein said pro-oncogenic factor comprises one of the group consisting of: a cancer stem cell (CSC) marker, a pro-oncogenic signaling pathway, a transcription factor, and a malignancy-promoting factor.

3. The recombinant oncolytic virus according to claim 1, wherein said recombinant oncolytic virus comprises a recombinant herpes simplex virus.

4. The recombinant oncolytic virus according to claim 3, wherein said recombinant herpes simplex virus comprises a recombinant herpes simplex virus-1 (HSV-1).

5. The recombinant oncolytic virus according to claim 3, wherein said viral gene controlling viral replication comprises a herpes alpha gene.

6. The recombinant oncolytic virus according to claim 5, wherein said herpes alpha gene comprises a gene selected from the group consisting of: infected cell protein-4 (ICP4), infected cell protein-6 (ICP6), infected cell protein 34.5 (ICP34.5), and infected cell protein-27 (ICP27).

7. The recombinant oncolytic virus according to claim 1, wherein said recombinant oncolytic virus comprises a recombinant virus selected from the group consisting of: adenovirus, vaccinia virus, vesicular stomatitis virus, poliovirus, reovirus, senecavirus, RIGVIR, Semliki Forest virus, measles virus, Newcastle disease virus, coxsackie virus, Maraba virus, and retrovirus.

8. The recombinant oncolytic virus according to claim 1, wherein said pro-oncogenic factor is selected from the group consisting of: CD133, Ezh2, CD24, CD34, CD38, CD117, CD44, CD90, CD271, ABCB5, EpCAM, JAK/STAT pathway, Wnt/β-catenin pathway, Hedgehog pathway, Notch pathway, TGF-β pathway, Ral pathway, Ras pathway, multidrug resistance pump ABC, CXCL12/CXCR4, VEGF/VEGFR, Cripto-1, Kpnb1, Enox-1, Enox-2, β3 integrin, Elk-1 binding factor, Oct4, sox2, nanog, Lin-29, KLf-4, c-myc, and combinations thereof.

9. The recombinant oncolytic virus according to claim 1, wherein said recombinant oncolytic virus is further coated with one of the group consisting of polymers, nanomers, and combinations thereof configured to improve efficiency of targeting said pro-oncogenic factor.

10. The recombinant oncolytic virus according to claim 1, wherein said recombinant oncolytic virus is further equipped with one of the group consisting of antibodies, aptamers, receptors, homing devices, and combinations thereof to improve efficiency of targeting said pro-oncogenic factor.

11. A recombinant herpes simplex virus configured for transcriptionally targeting cancer stem cells (CSCs) expressing a CSC marker, wherein:
a promoter for a herpes alpha gene is deleted;
a natural or synthetic sequence capable of driving expression of said herpes alpha gene and including a promoter for said CSC marker is inserted in controlling relation to said herpes alpha gene such that said promoter for said CSC marker controls expression of said herpes alpha gene;
said herpes alpha gene is only expressed within cells expressing said CSC marker; and
expression of said herpes alpha gene results in replication of said herpes simplex virus within and destruction of an infected cell.

12. The recombinant herpes simplex virus according to claim 11, wherein said recombinant herpes simplex virus comprises a recombinant herpes simplex-1 virus.

13. The recombinant herpes simplex virus according to claim 11, wherein said herpes alpha gene comprises infected cell protein-4 (ICP4).

14. The recombinant herpes simplex virus according to claim 11, wherein said CSC marker comprises CD133.

15. The recombinant herpes simplex virus according to claim 11, wherein said CSC marker comprises Ezh2.

16. A method of treating cancer in a subject comprising the steps of:
administering to a subject the recombinant oncolytic virus according to claim 1; and
said virus selectively killing tumor cells.

17. The method according to claim 16, wherein:
the tumor cells are selected from the group consisting of: hepatocellular carcinoma, colorectal carcinoma, melanoma, and glioma cells.

18. The method according to claim 16, wherein:
said administering to a subject the recombinant oncolytic virus is carried out by one of the group consisting of: injection, infusion, instillation, inhalation, and pharmaceutical ingestion.

19. The method according to claim 16, wherein:
said subject comprises a human.

20. The method according to claim 16, wherein:
said administering to a subject the recombinant oncolytic virus comprises administering said recombinant oncolytic virus in a preventative treatment to prevent development of malignancies.


SEQUENCE LISTING (30 sequences)

SEQ ID NO: 1 - DNA, 507 bp, Artificial Sequence
  based on Homo sapiens sequence
  misc_feature (133)..(133): n is a, c, g, or t
cctgtgtgtacacatgggagcgaatatggctttatgctgtttttcaaccgcttcagccactagcattggtactgctgcacactaaggatccaaatgattgtttttttgttttttgttttgttttgttttgttnttgttttgttttaaattgcattgggattaggcaacagaagggtctaatgcggccgggatgagacaggagagtttttaggagggtagctgcattctaagtaagggactctgcggggggaaaagaggcgcaagcgttgcaagaagggagtgcagggggttgagcaggcacctctacaggaaatggatgctgtccaggtgctggtgggcgccccagggctacgtggcgaagcagctcagccggtccaatcagagtgcgtccagggctcgggtttcgcgatctttaagtgactgaggcagatccccacactagtgctagcttgggtaacgccagggttttcccagtcacgacgttgtaaaacgacggccagtgccaag

SEQ ID NO: 2 - DNA, 1523 bp, Artificial Sequence
  based on Homo sapiens sequence
cagaggcggcaagtgaacagtgctcatcttgataaaggatttttaacactgacacgtgcttagaactacgaacagtggaagggtctgaaatggcagcaatgtcgccctttcacaggtttctagggcgataagcactgcaagctcggccagccaaagtccagcggtgggggaaaaaacggccaaggcagcagcagacataatcaaacagggcccgggtcggcggcggtggctcacgcctgtaatcccagcactttgggaggccgaggcgggcggatcaccagaagtcgggagttcgagaccagcctgaccaagacccgtctctactaaaaatagaaaattagccgggcgtggtggcgcatgcctgtaatccagctactcgggaggctgaggcatgagaatcgcttgaacccggaaggcggaggttgcggtgagtcgagatcgcgccattgcactccagcctggacaaccagagcgaaactccgtctcaggagaaaaaaaaaaaagtagtaacgggtccggcggcagcgcgcgggccgggcgagcgtctcccggcaaacgcggcgccacagctgagccgacctccggggcccgcgccctcccctccccgggcaccactaggagcggccagcccgggcctcggctccgcgcgcggggaaacgagcgcggcggttaaaaccgttaccacccccgagttttgaactggttcaaacttggcttccagcacccgccccgcccctcccccgcccgggaactctgcggcgccggttcccgccaagagccgccggcgcttcgtcccgcccttcggccggttcccgccacctatcctccccgcctcccgtccgcggcgggctccgggcccccgcgatgtctcccggtccccgcgtgcctgcacaccgccttcctgagaggcgccgtgtgttcagcgaaagaacaaagagacggcggcggcgcttccacacggccagtggcgtcccttacagcgaaccccgccgccgcccgcgcgcgcacgcgctgccagtgcccgcccgcccacgagccctgagcgcactctgcgtggggctggctcggcgcctccgagcccggcgggccctgtgattggacgggcgcccgcctcgcgtcccgccaatcggggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaaggtaacgcgccgctgcgggcggcccggccggcggggctccgggagtgcgaaccgggcggcggcggcggcgccaggacctccccgccactgctgtgccggtcccgggtatcgccgagcggggctcaccggggcgccgcgtttgtaggcgtgcggggggtggagggtgaggggagagcccccctccccggaaggagctgtgagcttcggg

SEQ ID NO: 3 - DNA, 2827 bp, Artificial Sequence
  based on Homo sapiens sequence
atggccctcgtactcggctccctgttgctgctggggctgtgcgggaactccttttcaggagggcagccttcatccacagatgctcctaaggcttggaattatgaattgcctgcaacaaattatgagacccaagactcccataaagctggacccattggcattctctttgaactagtgcatatctttctctatgtggtacagccgcgtgatttcccagaaggggcggcagcacaggaacccggcccatggcccatcgcactgtcggggagccccagcccagccgccccatcatcagggtggctgcactgcaatggtcaggatatcaggctccaggagatccctggagcgtcccttcaccctgtgcaacctgagtactcgcttcctctaaaggaactggaaatgataatcttcccagttacgggctgggctgtaccacatgctacatccagatactttgagaaaattcttacagaaggcatatgaatccaaaattgattatgacaagccagaaactgtaatcttaggtctaaagattgtctactatgaagcagggattattctatgctgtgtcctggggctgctgtttattattctgatgcctctggtggggtatttcttttgtatgtgtcgttgctgtaacaaatgtggtggagaaatgcaccagcgacagaaggaaaatgggcccttcctgaggaaatgctttgcaatctccctgttggtgatttgtataataataagcattggcatcttctatggttttgtggcaaatcaccaggtaagaacccggatcaaaaggagtcggaaactggcagatagcaatttcaaggacttgcgaactctcttgaatgaaactccagagcaaatcaaatatatattggcccagtacaacactaccaaggacaaggcgttcacagatctgaacagtatcaattcagtgctaggaggcggaattcttgaccgactgagacccaacatcatccctgttcttgatgagattaagtccatggcaacagcgatcaaggagaccaaagaggcgttggagaacatgaacagcaccttgaagagcttgcaccaacaaagtacacagcttagcagcagtctgaccagcgtgaaaactagcctgcggtcatctctcaatgaccctctgtgcttggtgcatccatcaagtgaaacctgcaacagcatcagattgtctctaagccagctgaatagcaaccctgaactgaggcagcttccacccgtggatgcagaacttgacaacgttaataacgttcttaggacagatttggatggcctggtccaacagggctatcaatcccttaatgatatacctgacagagtacaacgccaaaccacgactgtcgtagcaggtatcaaaagggtcttgaattccattggttcagatatcgacaatgtaactcagcgtcttcctattcaggatatactctcagcattctctgtttatgttaataacactgaaagttacatccacagaaatttacctacattggaagagtatgattcatactggtggctgggtggcctggtcatctgctctctgctgaccctcatcgtgattttttactacctgggcttactgtgtggcgtgtgcggctatgacaggcatgccaccccgaccacccgaggctgtgtctccaacaccggaggcgtcttcctcatggttggagttggattaagtttcctcttttgctggatattgatgatcattgtggttcttacctttgtctttggtgcaaatgtggaaaaactgatctgtgaaccttacacgagcaaggaattattccgggttttggatacaccctacttactaaatgaagactgggaatactatctctctgggaagctatttaataaatcaaaaatgaagctcacttttgaacaagtttacagtgactgcaaaaaaaatagaggcacttacggcactcttcacctgcagaacagcttcaatatcagtgaacatctcaacattaatgagcatactggaagcataagcagtgaattggaaagtctgaaggtaaatcttaatatctttctgttgggtgcagcaggaagaaaaaaccttcaggattttgctgcttgtggaatagacagaatgaattatgacagctacttggctcagactggtaaatcccccgcaggagtgaatcttttatcatttgcatatgatctagaagcaaaagcaaacagtttgcccccaggaaatttgaggaactccctgaaaagagatgcacaaactattaaaacaattcaccagcaacgagtccttcctatagaacaatcactgagcactctataccaaagcgtcaagatacttcaacgcacagggaatggattgttggagagagtaactaggattctagcttctctggattttgctcagaacttcatcacaaacaatacttcctctgttattattgaggaaactaagaagtatgggagaacaataataggatattttgaacattatctgcagtggatcgagttctctatcagtgagaaagtggcatcgtgcaaacctgtggccaccgctctagatactgctgttgatgtctttctgtgtagctacattatcgaccccttgaatttgttttggtttggcataggaaaagctactgtatttttacttccggctctaatttttgcggtaaaactggctaagtactatcgtcgaatggattcggaggacgtgtacgatgatgttgaaactatacccatgaaaaatatggaaaatggtaataatggttatcataaagatcatgtatatggtattcacaatcctgttatgacaagcccatcacaacattga

SEQ ID NO: 4 - DNA, 5766 bp, Artificial Sequence
  based on Homo sapiens sequence
gacagaccccagatagccttccagctgcgtgccaggtgctttacctattagatcgctagcctgcgaaccctatgcgaaatcctcctttgacagaggagcagaagaagggttggagaattcaaacaactgtcatcttggcagagttagtaagggggaccagagcccccttactgttgctgttgtctgtctattcagcagtgccctttagtttattcttgttttttttttcatgtgttccataatttttttctaaacttccttctgatttctaaacttttctaaacttctttctgatttctaaacttccttctgatttttatacgagacagtttctttgtgttttagtacaatgaggtgaacaaatgcatcagaggacaagctgaaaactttaaaccaagttggtactgtttaaaatataaaggaaataggtttttcagggagcaaagagctatttctgagatttgttaagggtcaagatactttgttaaaacagtggaagctgagtcgggggtaatttttatcagataaaggacttcttggattctgtaagttgcctgttcactctgatggtagtttcttttgctctgcagaagctctttagtttaattagatcccatttgtcaattttggcttttgttgccattgcttttggtgttttagacatgaagtccttgcccatgcctatgtcctgaatggtattgcctaggttttcttctagggtttttatggttttaggtctaacatttaagtttttaatccatcttgaattaatttttgtataaggtgtaaggaagggatccagtttcagctttctacatatggctagccaggtttcccagcaccgtttgttgggaattgaacaatgaaaacacttggacacaggaaggggaacatcacacaccggggcctgttgtcaggtggcgggagtggggagggatagcattaggacatatacctaatgtaaatgacgagttaataggtgcagcacaccgacatggcacatgtatacacatgtaacaaacatgcacattgtgcacatgtaccctagaacttaaagtataataaaaaaatatatatacatatacatatatatatgtatatgtatatatataaaaggacttcttgaaaggatggtccctgggtggcttaggtaagtggcagcaaggctggctagccaggcttaggaatctaggacatgccacaggacccctgatgggtgggtggctgcggctgccagaacaggaccctggatgtggctactggcattgctgccatcattgctggaatgaattagatactgtcctgcttcagagtgcgcaggcccaggcccagaagaaagcttctgattggcccagcatggctcagctccaccaggacacagtttgggcgcctcagctgacctcacaatgcaaggcagggctgcctcccacccagctcacaaacgtggtctcagatgttgggtatcccccatgcccccgccaaaagcactccagatgacaaaaatccactacaaagctcttctttaaaaagaaagattctgagaagttccatcaaaagaagaaaagaagaaatgaaggtgattgatcaccatatttgctttttcatttttaatttttttttttgagatggagtctctgtcacccaggctggagtgcagtggcatgatctcaggtcactgcaacctccgcctcccgggttcaagtgattctcctgcctcagcctcccgagtagctgggactacaggtgtgcaccaccacacctggctaattttttttgtatttttagtagagacagggtttcaccatgttagccaggatggtctcgatctcctgacctcgtgatccgcccgcctcggcctcccaaaatgctgggattacaggcttgagccaccacgcccgggccatatttgcttttaaaccagtattgaaatgcagttctctggactccaggctcaaaatcatgtaaggtcgcccaggctggagtgcagtggtatgattatggctcactacaggctcagcttcctggactcaagtaatcctcctgcttcagacttccgagtagctgggactacagttgtgcaccactacgtttgctaatttaaattttttttttttttttttttttttttgtagagacagggcctctctatgttgcccaggctggtagcaattcttcattgagtaccaactgtgttccaggctctggggatacagaaataagccagggacctgcacagcagctggggtagatcaattcaaagcaatatgaaaagggcatttggggagctttcaccttacatgtgttattttccaatgtgtttgggaagcctgaagtgtccttggaaagtgacatgaattgactaggcgggtactccaggatcagagtactccaggaacagaactgttctcaaactttgtttgaaggatcagtccccaggaaagcttgttaaaaatgcagattaatgggtttcatcccagaaattaggtccattaatagtctgcatttcaactaaacaccccattttatcccgagacagatgctactctcagtggctctcggacctagtggggtttagctggtaaatactcagagcttctgtttttggcacaacctattaaacccgagataaaaaactcttcctctttgacttgactttaatcagtttctagtatttcatagttagtgagatattaatactttcttaaaatgtactttaaaacttcagagggacctacattttggagcaagatgtaaagaaatctggaagtgcaactgcagttgctgtctatctcaagtcattgtctcccatctttgctgcggtgagcagttatgtttcacttcactgggaatttcccttcccctgctttgtcactgagggagttggagagcctgtttgttcatagttcttcccttggcagtttctctctgtcctgggctgaggtttggtggaattaccagaaagtgggccttctcagcatctgagcctttaatccttccagctgctgtgctggaggcttagctctctgcctgtcctttagcaaaaaaaaaaaccaagataaagtgtacatgtggtgtgttgtgcttgcagtagatgtttgaagggagccctttggaaacaccagggtcaaggtgtctattgtggatgtgacggtgggcgtaacctgaagcgtgggcctgaggttagtactggcagccacttgggatctgagcagcccatgatttgacaaaggaagaatttggggcccttgtattggtagctttgtgcaggagtaattggagtgggacaggacttagaagatgaggcaatggagggattagatatgaattaacatctttgttcaaatagctcaagaaatcccatctgttgtccaaagctttctttgacttggaatattggtagtgggtcctctggcttcttgcaaagtatgtcaccttcttcatcatcccccttccctttcttacagtatgtgtttcaggagcaccctgtacttatcttgaaagttagtaagtatgtatctagctcacaaagtgacagcagcttgctcttgctccaggacacttaatacagagaggaagagtggtacacttgggatccttccaggttctgtgctgatttgcctagtctcatgaaaccgcacaaggaaataacatattttggcttctacgccagcacttgccctgtagctctggagcaggctgtgtttggtttccacccacctacctggattaggctgaaggccctgctctaaagctttgcagcctcctgtctggggcacttcttgctgcaggcaggttacatgcattggtgactctgctgctatttctttccctgccagggatggtgccatgagtgaaacaagctgctcagcaggcacttactaaacaccgactacgtgccatgcccagtgttaaggtctggggattgaaagaagagtaggacagagtccatcttggaggcactttctgtcccattcccttcccagatgctggagccacagctgcttttaggattcttgtgtctttctccgtctcccaaggggtaggtaagagtctccctctgccctggctttcatctctgagacccctagttgtagaggtggtgacttagggtgaagatttccatcaggattacttggtaagcttattagaaagacaggttccagtgttgcacatgtgtccagcctcaaacggtgtggtccctagacttgaggccatgcccttctactttggattcggggtgaggtggagagggggatgtttgaaatgagattagtgagtaaataagtagaatataaggcagagtaagatatatctccagcggcaaagagaacaacacacacttggagaagaaataagcatataagcatgtactggattgctggggccccagtggctcgtactagagcctgagagggagggcactgaagggacagtgtcttgatgtcatagttagatcttggactttgaaggcaaactagtgggtttgactcttggccgcatggttaattgaaggggtgggttgcccctccacacctgtgggtgtttctcgttaggtggaacgagagacttggaaaagaaaaagacacagagacaaagtatagagaaagaaataagggggcccaggggaccagcgttcagcgtacggaggatccatcctggcctctgagttcccttagtatttattgatcattcttgggtgtttctcagagagggggatgtggcagggtcacaggataatagtggagagaaggtcagcagataaacacgtgaacaaaggtttctgcatcatagacaaggtaaagaattaagtgctgtgctttagatatgcatacccataaacatctcaatgccttacagagcagcattgttgcccgcatgtcccacctccagccctaaggcggttttcccctatctcagtagatggaacatacaatcgggttttataccgagacattccattgcccagggacgggcaggagacagatgccttcctcttgtctcaactgcaagaggcatgccttcctcttatactaatgctcctcagcacagaccctttacgggtgtcgggctgggggacagtcaggtctttcccttcccacgaggccatatttcagactatcacatggggagaaaccttggacaatacctggctttcctaggcagaggtccctgcggtcttccggccttccgcaatgtttgtgtccctgggtagttgagattagggagtggtgatgactcttaacgagcatgctgccttcaagcatctgtttaacaaagcacatcttgcacagcccttaatccatttaaccgtgagtggacacagcacatgtttcagagagcacggggttgggggtaaggttatagattaacagcatctcaagctgaagaatttctcttagtacagaacaaaatggagtctcctatgtctacttctttctacacagacacagtaacaatctgatctctctttcttttccccacattagggagggcactgaagggacagtgtcttgatgtcatagttagatcttggactttgaaggcaaactagtgggtttgactcttggccacatggttaatcacctgtgaccttgggcaagtcaccagaatcctctctgtgtcagtttcctcatctgcaaaatggaataatgccttaaacaaggaggaccttgtttctatctatgaagcatggagaacagtgcctggaagatagtaagtgctcaataaatgttagtttccaagatctcaagctttctctacaatatt

SEQ ID NO: 5 - DNA, 6600 bp, Artificial Sequence
  based on Homo sapiens sequence
gcatgtctgtctctgtgtccaaattttccctttttaaaaaggcactagccataatggattagggttcaccataatgagttcattttaactaaacaaattacctctataaacaccctgtctccaaataaggtcacattctctggtactggggattgggacttcaatgtatgagtgttttggggcacaccagttagcccgtaacagggtagctgcaatgatgacaggaaatcagggatgggtaagcattcataaatgtcactctcctagaggaaagggaggcttcctttcggggacattttttgtcctcatcttgcctgggttatagaagcaactcacataggatttttgctgggtgactccctaccctactgcagatgattccactgtacactctgggagggacatgtgctgtaaactcagcataaaatgtcatagtggtcccaactcagcataaaatgtcatagggcaccccggctgggtgaattcttccagaccctgttttctcttcctcatgcttcagacctcttcatttcacaagcagtctgttgaagtaaccaaactcccaggcccagagtcagccaatctgcatttcaaccccccacctccattcctcagcaatggcccttcatcttggaaatcagaattaatcacagcctctatgcctgaattaaagtttgctcttgtatcttctttaccaagtttacatatactcaatattggtgtatacaactatgtaatgcagtatgacctctctctgtctctgttctcctggcaactcattcaggctccattcccttcatatcccacttgaatttgcactactctgcaccactgactttagaatctgtcttgtaaatagggtcagagttcctcagccagatcccgatgggtaaaaggaaccatgtccccaccctgagttgagcaggtcttcaagaaaccctcttgtgagtccactctgccccaaagctgcctcccaaagatgcagcatctgtcctggcagagtgtgggcaggaaggctgccttgccaggctcctgactcccagtgctgcgtggtgctgatgagctgagctgatctttgaggggccagaccaggtgaaagttcccaccctaagcagctcagggcagggacagaccaaatccaagtgcaccaaatggggccatgagaaaccctcctgtggggatatctgtgatctcaggaatgaccctgagaggacactgctctgatgctctgatgagactgagggggattccaagcttccagggtgctgggcagtgtctccccagagagtggctgttcccagtgtcaatcaggcaggaagggtagaatgctgggacaggaagtagcttggaggtgggccttaggctggtagaagttgctgctttcttctctgtgggctcctttctcgtggatctggaccccaggagttcccaggcatagagaggaggggtgccaggtgatgggggtgatggaggcggtggccacggccctggagaaggctgagccaggcaggggacagctatgggcagccttgaggttctgagagcagccgtgggcagcagtgggcagttgtcagttactctgtttatgtttaggtcttctgtaaaatcttgctaaacaatcgggtttgatctcatttgggttggggcaaattgagggaagagtctgtgttcaggtatagatcagagtttaaagaacaggcaagcctcaccatgccagggatacgatgacactagtgaggtccccgagggaaaggccctcccatggtggagaatctccctggctgctgccaaccttggagaatcgaggggagcatttgatcaggatgccagtgagggaggcagagccaccccagtgactgaagcccttctcagtgggttttctgcccaaagactggtcaaggattgtgtgccgagggtcaggggtgtttgtaaggacagagggagccagcagggagccaagagaatctgtcccagaaagcctaggtttaaactccagactccaagagtcagagcaggagcagagagtgggttacagaacttggggttgggaaaccttatcagtgttcttccatatctctttggtctcatttcctagccctgcgttggacccatccaggcatctttgctgttgcctgagcacatgaagccggcccctttttcccagcctttgcactgtactttgcctggaacgtgcttcccttgggtattcactttactcaagtcctctcaaatatctgagagagaggtctctgaccaccccatggaaaagaacaactcttacctatcccttacacattctcttactacacaacactaatttcctacaattatattctacagtatgtttatttgtttctctctttattgtctgtttgccacagtggagtgtaagcaccatgacaacaggaactgtgtcttgaccactgttgacttccaagcacctcaaataggacctgtcacatagaagtttcaatcaatatttgttgagtgaatgaagacaacaggtgtcaacttagcttctactacagttctaagacttttgcgtcaagaagtggggacactccagatgcaatggtgaaaagccacgattatttccacctggccctttctcctatctgtcccgctatctttctgttacttgctcatttctcccaagatctgtcccttcagaggattctttgactccactccatacatttattcttccttgctgtgctcttgctgttgcatcttactgttccctcttcattctcagctttccaaaatcttactcctctttaaggccagaacttattcatccttcaagggctaactgaaggctctgtaaccttgtttgtaaaatcataccaggtgaaactaatattccttttccttggatcccagaaccttttcttgatagtttgattaaggcatttttacaatctgatgtaattaagacataaaaaaggacaaaggctctatcctccctggaagatagggaggttttttggtttgtttgtttgtttttgcttttgttttctttttttgagacagggtctcactctgtcactctgtcacccaggctggagtaaagtggggcatgatcacagctcactgcagtctcaaattatgagctccagcatcatccctactcagcctcctgagtagctgggactacaggcacaagcctgtctaagttttttcttatttttttaacagagacaaggtctccatatgttgcccaggcttgtcttgaactcctggcctcaaacgagcctcccgcctcagcctccctgggattacaggtgcaagccacagcacctggctaggattcttacaggcatgaatgaagtgctggtatctctagtaatttgcacataacaaatgcccaattaatgtttgcttaattgaataaaattgacattattctctgagaattaggtggggaaaatgaaatgaatttgaatgtgtgctgtatgctatacactgtggtaataatgcgataatatactttatttccaaaaaccctcccagcaatcttactgaagtgtattaccatccctgttttgcagaggatggtcagagaagttatgtgagctgcccagggttacacggctactgacaggcagaaccagggtttgaagtcagatttatatatttaaagacaaaatggcttatactcattaaaattatttttatccctacagattcaagatgggattctagtagacagcatagctacacatcaggcatagatttggaagcctggttttaacatgccaaagggtaaataaaagccatgtgtcctttctccatgaggatgagaatacttttatatagtctttttgtgtggagagagtctaaactacacagagaactaccccaggtgaatcaaaacaattgactacggtgaggtcacataaattagtgatacctagttaactttttgtcttgaggccagagctagtgttggctcacttgggacttgagaaggcaagagaggtatttatagcaacagtgacagctgactgcaagtattcctatgacgattaaatttattctaggaaatcttataaatagtgagtttaatagctgtcactttgagttcatcagctgaagcaacagtttagaattcatatttgaaaacctccattgaagctaaaatgtagaaaaatagagggctaccctggtgtggtatctcagggcaaatgtagctcactgaatttagcttggtccctaaagaagtaaaactttacaatagttaagtgaaccaaaatctttgccatatcaaaactaatcgtaaaaaggcatctttccaatggatgcagcttgccgtttaatagaatatatcagagcatgaaaattgtcctttaaagcatgtccactaactccttcttcaactaaatgcctccagagagaaaatgaatgcattctaaaactttcatgctttttttccccagaaaagctgattcccaaaatatctaacctataaactttgaacaatagcaacccttttaaaaaccctaaaaaactatataaagaagggctggatggagctacatttatatatttacagttatttgtttttcttaaagttttcctcttgatgataattaaatttaaatgtttttttcatagaaaatctggaaaatacacaaaaattaaatagagaagaaaaaagaaaaagatttttgccccactgctcatagaggcaatcatggttaacgtgttgatgtattttcttttccctaagcttggttttcatagtccttggctttgctgttatttatttgctcatcatttgttctcaacagtgccaacgacctcacagacacaggttgtggatagcagcagacatggaggtggaggctgggcggccagtgatgctgtgagtcaggtgagtcatttctcatctctaggccagacgttttcacaggttggttctcagaatctgagaaaagcagcagaccctctttccacaaatatccacaatagacccacgctttgcatacaagttcagggcccacatccaaatcccttagcgatccaggagcctctcaggttaaacatcttgaactaggtatcagactccttgacagtttgcaatgctgccttctcaaagaagcttgcaaatgtctccatttattatttgagtcaatgtgagttgtacgtaaagaaagaatgaggagaaagccctctcagatggtcatctgggcctttctactacaaacagccttgctaactctgggaatctcaagccatgctataggaagtggggaggaaagtaggcttctgtgcatgatcccaagtccacactgccagagacatccagatgagcacagtctatttgtgctggggtcagtggagaagaacctgttagtcaaggctcaaggaaggctttgttttttctgtccctgcacaatggtcctgattcattacgtgctctggcatgcagttggtgtcacatgggtgtgatgaccacctctaggttctgatcacacagggtccagaggaaccagagcaatgactggatctgagaggcaccagcttcccaaccatcccaacgcactgtacagtaactgcacacccccagccctgccactcccccgtccccccaacatctccttccctagggacacagctaagctaacacaagataaatgcaaacaacaagctcagtcaaggacagagccaggtgctggcaggcagggcagagggtttagtgcagtcagctggggacccaggggtgactgatgtcctgcaaataacttctaattgtaagtctggaagggaggatgcaatagaaagaaatgaggaagcttctactcactgaccacgtactgcaactagtgctatgtgtatagtaattctattgttcaagcttttcaataaacattcaaggtaggtattgttcagccattgacccaacaatattattggctggtggctctgtgccagaccatggggagagcagaagcaaactgacccaagttcctgctgccatggagcttaccttctaatgggcaagcattcaggaaagaattgggcaaatgcgtgtaagtgatgacaggtgtgagtgcaccaaaggaaagctgagctgctcggtcagcgagtggcaggcagtcgagaaaggcctggggagtgagggagctgtggaggtgtctggggcttacccctctgcccccaggcatggggagcagtcagtgcaaaggtctcgagaggcaggctgcctgatgttgagggaggccagcgcgactggtgtccagttagaaaggggaaggtgtgggtgttggggcttaggacagatagttacaagccacactgggcactttgcacattgtgctgagtaaggaatgagatagtgtgttaatctgtattgtgttgctctaaagaaatacctgagactgggtaatttataaaggtttatttggttcatagttctgcaggctgtacgggaagcacggcaccagtatctgcttctggtgaggactcaggagcttaaaatcatggtggaagcagggagagcaagcatgtcacaaggtgagagaggaagcaagagagaagtggagaggtcccagactctttctaacaaccagatctcacatgaattccttactgctg

SEQ ID NO: 6 - DNA, 33 bp, Artificial Sequence
  based on Homo sapiens sequence
ctcacagctagcttgggtaacgccagggttttc

SEQ ID NO: 7 - DNA, 34 bp, Artificial Sequence
  based on Homo sapiens sequence
actgtatagatctcgcccctcgaataaacaacgc

SEQ ID NO: 8 - DNA, 26 bp, Artificial Sequence
  based on Homo sapiens sequence
atcgctagctccaagactgacacatt

SEQ ID NO: 9 - DNA, 50 bp, Artificial Sequence
  based on Homo sapiens sequence
atgctagcactagtaccggtagtactgctgaggtgggctttggacgtctt

SEQ ID NO: 10 - DNA, 36 bp, Artificial Sequence
  based on Homo sapiens sequence
gggttaacatttaaatcaggtcgccgttgggggcca

SEQ ID NO: 11 - DNA, 27 bp, Artificial Sequence
  based on Homo sapiens sequence
gggttaacaaatgagtcttcggacctc

SEQ ID NO: 12 - DNA, 20 bp, Artificial Sequence
  based on Homo sapiens sequence
aaactgtctttcctggcttc

SEQ ID NO: 13 - DNA, 20 bp, Artificial Sequence
  based on Homo sapiens sequence
ttccttaaacatactcaccg

SEQ ID NO: 14 - DNA, 30 bp, Artificial Sequence
  based on Homo sapiens sequence
tgaaccggtcctgcaagcggcacatcagag

SEQ ID NO: 15 - DNA, 32 bp, Artificial Sequence
  based on Homo sapiens sequence
tgaactagtgcgttagcatcgctttaattcag

SEQ ID NO: 16 - DNA, 22 bp, Artificial Sequence
  based on Homo sapiens sequence
tgcagaggcggcaagtgaacag

SEQ ID NO: 17 - DNA, 22 bp, Artificial Sequence
  based on Homo sapiens sequence
ttctgagtcccaccgggtgtcg

SEQ ID NO: 18 - DNA, 23 bp, Artificial Sequence
  based on Homo sapiens sequence
taccggtgaactacgaacagtgg

SEQ ID NO: 19 - DNA, 24 bp, Artificial Sequence
  based on Homo sapiens sequence
tactagtcggaccgagcgccaaac

SEQ ID NO: 20 - DNA, 27 bp, Artificial Sequence
  based on Homo sapiens sequence
cagtacggccccgagttcgtgaccggg

SEQ ID NO: 21 - DNA, 24 bp, Artificial Sequence
  based on Homo sapiens sequence
ggcgtagtaggcggggatgtcgcg

SEQ ID NO: 22 - DNA, 20 bp, Artificial Sequence
  based on Homo sapiens sequence
tggattgcacgcaggttctc

SEQ ID NO: 23 - DNA, 20 bp, Artificial Sequence
  based on Homo sapiens sequence
gatcgacaagaccggcttcc

SEQ ID NO: 24 - DNA, 20 bp, Artificial Sequence
  based on Homo sapiens sequence
ggtcgtaatccaggataaag

SEQ ID NO: 25 - DNA, 20 bp, Artificial Sequence
  based on Homo sapiens sequence
tagatctaagcttggcactg

SEQ ID NO: 26 - DNA, 19 bp, Artificial Sequence
  based on Homo sapiens sequence
gtaatccagctactcggga

SEQ ID NO: 27 - DNA, 20 bp, Artificial Sequence
  based on Homo sapiens sequence
gctccaaggtgtacgtgaag

SEQ ID NO: 28 - DNA, 19 bp, Artificial Sequence
  based on Homo sapiens sequence
gctacaggaacaggtggtg

SEQ ID NO: 29 - DNA, 20 bp, Artificial Sequence
  based on Homo sapiens sequence
acggcaacaacctgattaag

SEQ ID NO: 30 - DNA, 20 bp, Artificial Sequence
  based on Homo sapiens sequence
gggacacgttatttaccctg